(19) United States
(12) Patent Application Publication
(10) Pub. No.: US 2020/0109214 A1
(43) Pub. Date: Apr. 9, 2020

(54) PLASMA KALLIKREIN INHIBITORS AND USES THEREOF FOR TREATING HEREDITARY ANGIOEDEMA ATTACK

(71) Applicant: Dyax Corp., Lexington, MA (US)

(72) Inventors: Peng Lu, Lexington, MA (US); Christina Nurse, Lexington, MA (US); Xinming Hao, Lexington, MA (US)

(73) Assignee: Dyax Corp., Lexington, MA (US)

(21) Appl. No.: 16/556,524

(22) Filed: Aug. 30, 2019

Related U.S. Application Data
(60) Provisional application No. 62/808,612, filed on Feb. 21, 2019.
(60) Provisional application No. 62/725,216, filed on Aug. 30, 2018.

(51) Int. Cl. C07K 16/40; A61K 47/02; A61K 47/12; A61K 47/22; A61K 47/26; A61P 29/00

(52) U.S. Cl. CPC C07K 16/40; A61K 47/02; A61K 47/12; A61K 2039/545; A61K 47/26; A61P 29/00; C07K 2317/565; A61K 47/22

(57) ABSTRACT

Provided herein are methods of treating and preventing hereditary angioedema attack in certain human patient subpopulations using antibodies binding to active plasma kallikrein with specific treatment regimens, for example, at about 300 mg every two weeks. Exemplary human patient subpopulations include female patients, patients less than 18 years old, between 40 and less than 65 years old, adolescent patients, patients who have had one or more prior laryngeal attacks, patients who have had between 1 and 2, 2 and 3, or more than 3 HAE attacks in the four weeks prior to the first dose of the first treatment period; and/or has received treatment with a C1-inhibitor prior to the first treatment period.

### RELATED APPLICATIONS

This application claims the benefit under 35 U.S.C. § 119(e) of U.S. provisional application No. 62/725,216, filed Aug. 30, 2018, and U.S. provisional application No. 62/808,612, filed Feb. 21, 2019, each of which is incorporated by reference herein in its entirety.

### BACKGROUND

Plasma kallikrein is a serine protease component of the contact system and a potential drug target for different inflammatory, cardiovascular, infectious (sepsis) and oncology diseases (Sainz I. M. et al., Thromb Haemost 98, 77-83, 2007). The contact system is activated by either factor XIIa upon exposure to foreign or negatively charged surfaces or on endothelial cell surfaces by prolylcarboxypeptidases (Sainz I. M. et al., Thromb Haemost 98, 77-83, 2007). Activation of the plasma kallikrein amplifies intrinsic coagulation via its feedback activation of factor XII and enhances inflammation via the production of the proinflammatory nonapeptide bradykinin. As the primary kininogenase in the circulation, plasma kallikrein is largely responsible for the generation of bradykinin in the vasculature. A genetic deficiency in the C1-inhibitor protein (C1-INH), the major natural inhibitor of plasma kallikrein, leads to hereditary angioedema (HAE). Patients with HAE suffer from acute attacks of painful edema often precipitated by unknown triggers (Zuraw B. L. et al., N Engl J Med 359, 1027-1036, 2008).

### SUMMARY

Provided herein are regimens for treating hereditary angioedema (HAE) attack, reducing the rate of HAE attack, or blocking HAE attack using antibodies capable of binding and inhibiting human plasma kallikrein (pKal) in the active form, for example, antibodies having the same complementarity determining regions (CDRs) as DX-2930 (a.k.a. SHP643, lanadelumab).

In some aspects, the present disclosure provides methods for treating hereditary angioedema (HAE) attack or reducing the rate of HAE attack, comprising administering (e.g., subcutaneously) to a human subject in need thereof any of the antibodies described herein (e.g., DX-2930). In some embodiments, the antibody is administered to the subject in multiple doses of about 300 mg every two weeks in a first treatment period. In some embodiments, the subject has, is suspected of having, or is at risk for HAE and is female; less than 18 years old or between the ages of 40-65 years old; and/or has experienced at least one prior laryngeal HAE attack.

In some aspects, the present disclosure provides methods for treating hereditary angioedema (HAE) attack or reducing the rate of HAE attack, comprising administering (e.g., subcutaneously) to a human subject in need thereof any of the antibodies described herein (e.g., DX-2930). In some embodiments, the antibody is administered to the subject at about 150 mg every four weeks, at about 300 mg every four weeks, or at about 300 mg every two weeks. In some embodiments, the subject is an adolescent between the age of 12 and 18.

Any of the methods described herein may further comprise administering to the subject the antibody for a second treatment period after the first treatment period. In some embodiments, the first dose of the second treatment period is about two weeks after the last dose of the first treatment period. In some embodiments, the second treatment period comprises one or more doses of the antibody at about 300 mg. In some embodiments, the second treatment period comprises multiple doses of the antibody at about 300 mg every two weeks.

Any of the methods described herein may further comprise (a) administering to the human subject the antibody at a single dose of about 300 mg after the first treatment period; and (b) further administering to the subject the antibody at one or more doses of about 300 mg, if the subject experiences an HAE attack after (a). In some embodiments, in step (b), the subject is administered the antibody for multiple doses at about 300 mg every two weeks. In some embodiments, the first dose of step (b) is within one week after the HAE attack. In some embodiments, the single dose of (a) and the first dose of (b) are at least 10 days apart.

In any of the methods described herein, the human subject may have HAE type I or type II. For example, the subject may have experienced at least two HAE attacks per year prior to the first treatment period. In some embodiments, the subject has had at least one HAE attack in the four weeks prior to the first dose of the first treatment period or at least two HAE attacks in the eight weeks prior to the first dose of the first treatment period.

In some embodiments, the subject to be treated by any of the methods described herein, which involve the use of any of the anti-pKal antibodies described herein (e.g., DX-2930) have received one or more HAE treatments prior to the first dose of the anti-pKal antibody. Such prior HAE treatments may involve a C1-inhibitor (e.g., C1-INH), a plasma kallikrein inhibitor (e.g., ecallantide), a bradykinin receptor antagonist (e.g., icatibant), an androgen (e.g., danazol), an anti-fibrinolytic agent (e.g., tranexamic acid), or a combination thereof. Such a subject may undergo a tapering period to gradually transit from the prior HAE treatment to the anti-pKal antibody treatment described herein. In some examples, the tapering period is about 2-4 weeks. The prior HAE treatment may terminate either before the first dose of the antibody or within three weeks after the first dose of the antibody to the subject. Alternatively, the subject may be directly transitioned from any of the prior HAE treatments to the anti-pKal antibody treatment as described herein.

In some embodiments, subject has not received an HAE treatment prior to the first dose of the anti-pKal antibody. In some embodiments, the subject is free of prior HAE treatment at least two weeks before the first dose of the antibody.

In some embodiments, the subject is free of a long-term prophylaxis for HAE, or an HAE treatment involving an angiotensin-converting enzyme (ACE) inhibitor, an estrogen-containing medication, or an androgen prior to the first treatment period, during the first treatment period, and/or during the second treatment period.

In some embodiments, the antibody is a full length antibody or an antigen-binding fragment thereof. In some examples, the antibody comprises a heavy chain variable region set forth by SEQ ID NO: 3 and/or a light chain variable region set forth by SEQ ID NO: 4. In some examples, the antibody comprises a heavy chain set forth by SEQ ID NO: 1 and a light chain set forth by SEQ ID NO: 2.

In any of the methods described herein, the antibody can be formulated in a pharmaceutical composition comprising a pharmaceutically acceptable carrier. In some embodiments, the pharmaceutically composition comprises sodium phosphate, citric acid, histidine, sodium chloride, and polysorbate 80. In one example, the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

The details of one or more embodiments of the invention are set forth in the description below. Other features or advantages of the present invention will be apparent from the following drawing and detailed description of several embodiments, and also from the appended claims.

### BRIEF DESCRIPTION OF THE DRAWINGS

FIGS. 1A-1C include plots of the Poisson regression of investigator-confirmed HAE attacks during the treatment period (days 0-182) for patients based on the number of HAE attacks during the run-in period. FIG. 1A: 1 to <2 HAE attacks per month in the run-in period. FIG. 1B: 2 to <3 HAE attacks per month in the run-in period. FIG. 1C: ≥3 HAE attacks per month in the run-in period.

FIGS. 2A-2B include diagrams showing HAE attack rates in patients who previously received long term prophylaxis with C1-inhibitor (C1-INH). FIG. 2A: mean (standard deviation) historical (3 month), baseline, and during lanadelumab treatment (days 0-182) HAE attack rates per month. FIG. 2B: reduction in HAE attack rates in HAE patients each of the indicated lanadelumab treatment groups.

FIGS. 3A-3C includes plots of the monthly HAE attack rate in adolescent subjects. FIG. 3A: shows a plot of the estimated least square means (LS) monthly attack rate versus placebo for adolescent patients with 95% confidence interval. FIG. 3B: a plot of the monthly HAE attack rate during the period of treatment with lanadelumab versus baseline for rollover and non-rollover adolescent subjects. FIG. 3C: shows a plot of the estimated least squares mean monthly attack rate ratio (versus placebo), with 95% confidence interval, for adolescent patients in each of the indicated lanadelumab treatment groups.

FIGS. 4A-4E shows plots of the HAE attack rate percentage reductions, with 95% confidence interval, from placebo for each of the indicated demographics. FIG. 4A: age; FIG. 4B: sex; FIG. 4C: weight; FIG. 4D: HAE type; FIG. 4E: history of laryngeal attacks. For each of the indicated groups, the columns correspond to, from left to right, 150 mg every 4 weeks, 300 mg every 4 weeks, and 300 mg every 2 weeks. “n” below the plot refers to the number of subjects in each group.

FIG. 5 shows a Forest plot of the rate ratio of the number of investigator-confirmed HAE attacks based on the indicated demographic.

### DETAILED DESCRIPTION

### Definitions

For convenience, before further description of the present invention, certain terms employed in the specification, examples and appended claims are defined here. Other terms are defined as they appear in the specification.

The singular forms “a”, “an”, and “the” include plural references unless the context clearly dictates otherwise.

As used herein, the term “about” refers to a particular value +/−5%. For example, an antibody at about 300 mg includes any amount of the antibody between 285 mg-315 mg.

The term “antibody” refers to an immunoglobulin molecule capable of specific binding to a target, such as a carbohydrate, polynucleotide, lipid, polypeptide, etc., through at least one antigen recognition site located in the variable region of the immunoglobulin molecule. An antibody may include at least one heavy (H) chain that comprises a heavy chain immunoglobulin variable domain (V_{H}), at least one light chain that comprises a light chain immunoglobulin variable domain (V_{L}), or both. For example, an antibody can include a heavy (H) chain variable region (abbreviated herein as V_{H} or HV) and a light (L) chain variable region (abbreviated herein as V_{L} or LV). In another example, an antibody includes two heavy (H) chain variable regions and two light (L) chain variable regions.

As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), domain antibody (dAb) fragments (de Wildt et. al., Euro. J. Immunol. (1996) 26(3): 629-639), any mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Antibodies may be from any source, but primate (human and non-human primate) and primatized are preferred.

The V_{H} and/or V_{L} regions may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form an antigen binding site, e.g., a structure that preferentially interacts with plasma kallikrein.

The V_{H} and V_{L} regions can be further subdivided into regions of hypervariability, termed “complementarity determining regions” (“CDRs”), interspersed with regions that are more conserved, termed “framework regions” (“FRs”). The extent of the framework region and CDRs have been defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

In addition to the V_{H} or V_{L} regions, the heavy chain or light chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3. The light chain constant region includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The light chains of the immunoglobulin may be of type kappa or lambda. In one embodiment, the antibody is glycosylated. An antibody can be functional for antibody-dependent cytotoxicity and/or complement-mediated cytotoxicity.

One or more regions of an antibody can be human or effectively human. For example, one or more of the variable regions can be human or effectively human. For example, one or more of the CDRs can be human, e.g., HC CDR1, HC CDR2, HC CDR3, LC CDR1, LC CDR2, and/or LC CDR3. Each of the light chain (LC) and/or heavy chain (HC) CDRs can be human. HC CDR3 can be human. One or more of the framework regions can be human, e.g., FR1, FR2, FR3, and/or FR4 of the HC and/or LC. For example, the Fc region can be human. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. In one embodiment, the framework (FR) residues of a selected Fab can be converted to the amino-acid type of the corresponding residue in the most similar primate germline gene, especially the human germline gene. One or more of the constant regions can be human or effectively human. For example, at least 70, 75, 80, 85, 90, 92, 95, 98, or 100% of an immunoglobulin variable domain, the constant region, the constant domains (CH1, CH2, CH3, and/or CL1), or the entire antibody can be human or effectively human.

An antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the many immunoglobulin variable region genes. Full-length immunoglobulin “light chains” (about 25 KDa or about 214 amino acids) are encoded by a variable region gene at the NH2-terminus (about 110 amino acids) and a kappa or lambda constant region gene at the COOH-terminus. Full-length immunoglobulin “heavy chains” (about 50 KDa or about 446 amino acids), are similarly encoded by a variable region gene (about 116 amino acids) and one of the other aforementioned constant region genes, e.g., gamma (encoding about 330 amino acids). The length of human HC varies considerably because HC CDR3 varies from about 3 amino-acid residues to over 35 amino-acid residues.

The term “antigen-binding fragment” of a full length antibody refers to one or more fragments of a full-length antibody that retain the ability to specifically bind to a target of interest. Examples of binding fragments encompassed within the term “antigen-binding fragment” of a full length antibody and that retain functionality include (i) a Fab fragment, a monovalent fragment consisting of the V_{L}, V_{H}, C_{L} and CH1 domains; (ii) a F(ab′)₂ fragment, a bivalent fragment including two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those with skill in the art.

The term “monospecific antibody” refers to an antibody that displays a single binding specificity and affinity for a particular target, e.g., epitope. This term includes a “monoclonal antibody” or “monoclonal antibody composition,” which as used herein refers to a preparation of antibodies or fragments thereof of single molecular composition, irrespective of how the antibody was generated. Antibodies are “germlined” by reverting one or more non-germline amino acids in framework regions to corresponding germline amino acids of the antibody, so long as binding properties are substantially retained.

The inhibition constant (Kᵢ) provides a measure of inhibitor potency; it is the concentration of inhibitor required to reduce enzyme activity by half and is not dependent on enzyme or substrate concentrations. The apparent Kᵢ (K_{i,app}) is obtained at different substrate concentrations by measuring the inhibitory effect of different concentrations of inhibitor (e.g., inhibitory binding protein) on the extent of the reaction (e.g., enzyme activity); fitting the change in pseudo-first order rate constant as a function of inhibitor concentration to the Morrison equation (Equation 1) yields an estimate of the apparent Kᵢ value. The Kᵢ is obtained from the y-intercept extracted from a linear regression analysis of a plot of K_{i,app} versus substrate concentration.

$$v = {v}_{o} - {v}_{o} ( \frac{( {K}_{i , app} + I + E ) - \sqrt{{( {K}_{i , app} + I + E )}^{2} - 4 \cdot I \cdot E}}{2 \cdot E} ) \qquad Equation 1$$

Where v=measured velocity; v0=velocity in the absence of inhibitor; K_{i,app}=apparent inhibition constant; I=total inhibitor concentration; and E=total enzyme concentration.

As used herein, “binding affinity” refers to the apparent association constant or K_{A}. The K_{A} is the reciprocal of the dissociation constant (K_{D}). A binding antibody may, for example, have a binding affinity of at least 105, 106, 107, 108, 109, 1010 and 1011 M-1 for a particular target molecule, e.g., plasma kallikrein. Higher affinity binding of a binding antibody to a first target relative to a second target can be indicated by a higher K_{A} (or a smaller numerical value K_{D}) for binding the first target than the K_{A} (or numerical value K_{D}) for binding the second target. In such cases, the binding antibody has specificity for the first target (e.g., a protein in a first conformation or mimic thereof) relative to the second target (e.g., the same protein in a second conformation or mimic thereof; or a second protein). Differences in binding affinity (e.g., for specificity or other comparisons) can be at least 1.5, 2, 3, 4, 5, 10, 15, 20, 30, 40, 50, 70, 80, 90, 100, 500, 1000, 10,000 or 10⁵ fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl, 0.005% (v/v) Surfactant P20). These techniques can be used to measure the concentration of bound and free binding protein as a function of binding protein (or target) concentration. The concentration of bound binding protein ([Bound]) is related to the concentration of free binding protein ([Free]) and the concentration of binding sites for the binding protein on the target where (N) is the number of binding sites per target molecule by the following equation:

[Bound]=N·[Free]/((1/KA)+[Free]).

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2 fold higher, to obtain a qualitative measurement of affinity, or to obtain an inference of affinity, e.g., by activity in a functional assay, e.g., an in vitro or in vivo assay.

The term “binding antibody” (or “binding protein” used interchangeably herein) refers to an antibody that can interact with a target molecule. The term “target molecule” is used interchangeably with “ligand.” A “plasma kallikrein binding antibody” refers to an antibody that can interact with (e.g., bind) plasma kallikrein, and includes, in particular, antibodies that preferentially or specifically interact with and/or inhibit plasma kallikrein. An antibody inhibits plasma kallikrein if it causes a decrease in the activity of plasma kallikrein as compared to the activity of plasma kallikrein in the absence of the antibody and under the same conditions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

It is possible for one or more framework and/or CDR amino acid residues of a binding protein to include one or more mutations (for example, substitutions (e.g., conservative substitutions or substitutions of non-essential amino acids), insertions, or deletions) relative to a binding protein described herein. A plasma kallikrein binding protein may have mutations (e.g., substitutions (e.g., conservative substitutions or substitutions of non-essential amino acids), insertions, or deletions) (e.g., at least one, two, three, or four, and/or less than 15, 12, 10, 9, 8, 7, 6, 5, 4, 3, or 2 mutations) relative to a binding protein described herein, e.g., mutations which do not have a substantial effect on protein function. The mutations can be present in framework regions, CDRs, and/or constant regions. In some embodiments, the mutations are present in a framework region. In some embodiments, the mutations are present in a CDR. In some embodiments, the mutations are present in a constant region. Whether or not a particular substitution will be tolerated, i.e., will not adversely affect biological properties, such as binding activity, can be predicted, e.g., by evaluating whether the mutation is conservative or by the method of Bowie, et al. (1990) Science 247:1306-1310.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

An “epitope” refers to the site on a target compound that is bound by a binding protein (e.g., an antibody such as a Fab or full length antibody). In the case where the target compound is a protein, the site can be entirely composed of amino acid components, entirely composed of chemical modifications of amino acids of the protein (e.g., glycosyl moieties), or composed of combinations thereof. Overlapping epitopes include at least one common amino acid residue, glycosyl group, phosphate group, sulfate group, or other molecular feature.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762.

An “isolated” antibody refers to an antibody that is removed from at least 90% of at least one component of a natural sample from which the isolated antibody can be obtained. Antibodies can be “of at least” a certain degree of purity if the species or population of species of interest is at least 5, 10, 25, 50, 75, 80, 90, 92, 95, 98, or 99% pure on a weight-weight basis.

The methods described herein involve administering multiple doses of an antibody to a human subject in need thereof. The terms “patient,” “subject” or “host” may be used interchangeably. A subject may be a subject that has undergone a prior treatment for HAE, such as a treatment involving an antibody described herein. In some embodiments, the subject is a pediatric subject (e.g., an infant, child, or adolescent subject). In some embodiments, the human subject is an adolescent less than 18 years old. In some embodiments, the human subject is an adolescent between the ages of 12 and 18 years old. In some embodiments, the subject is between the ages of 40 and less than 65 years old.

In some embodiments, the human subject is defined by gender. For example, in some embodiments, the subject is female.

In some embodiments, the human subject is defined by weight. In some embodiments, the human subject weighs less than 50 kg. In some embodiments, the human subject weighs between 50 kg and 75 kg. In some embodiments the human subject weighs between 75 kg and 100 kg. In some embodiments, the human subject weighs 100 kg or more.

In some embodiments, the human subject is defined by prior history of laryngeal attacks or absence thereof. In some embodiments, the subject has experienced at least one (e.g., 1, 2, 3, 4, 5, or more) laryngeal attack (i.e. laryngeal HAE attack) prior to administration of the antibodies described herein. In some embodiments, the subject has not experienced a laryngeal attack prior to administration of the antibodies described herein.

The terms “prekallikrein” and “preplasma kallikrein” are used interchangeably herein and refer to the zymogen form of active plasma kallikrein, which is also known as prekallikrein.

As used herein, the term “substantially identical” (or “substantially homologous”) is used herein to refer to a first amino acid or nucleic acid sequence that contains a sufficient number of identical or equivalent (e.g., with a similar side chain, for example, conserved amino acid substitutions) amino acid residues or nucleotides to a second amino acid or nucleic acid sequence such that the first and second amino acid or nucleic acid sequences have (or encode proteins having) similar activities, e.g., a binding activity, a binding preference, or a biological activity. In the case of antibodies, the second antibody has the same specificity and has at least 50%, at least 25%, or at least 10% of the affinity relative to the same antigen.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular binding proteins may show a difference, e.g., in specificity or binding that are statistically significant (e.g., P value<0.05 or 0.02). The terms “induce”, “inhibit”, “potentiate”, “elevate”, “increase”, “decrease” or the like, e.g., which denote distinguishable qualitative or quantitative differences between two states, may refer to a difference, e.g., a statistically significant difference, between the two states.

A “therapeutically effective dosage” preferably modulates a measurable parameter, e.g., plasma kallikrein activity, by a statistically significant degree or at least about 20%, more preferably by at least about 40%, even more preferably by at least about 60%, and still more preferably by at least about 80% relative to untreated subjects. The ability of a compound to modulate a measurable parameter, e.g., a disease-associated parameter, can be evaluated in an animal model system predictive of efficacy in human disorders and conditions. Alternatively, this property of a composition can be evaluated by examining the ability of the compound to modulate a parameter in vitro.

The term “treating” as used herein refers to the application or administration of a composition including one or more active agents to a subject, who has HAE, a symptom of HAE, is suspected of having HAE, or a predisposition toward or risk of having HAE, with the purpose to cure, heal, alleviate, relieve, alter, remedy, ameliorate, improve, or affect the disease, the symptoms of the disease, or the predisposition toward the disease. “Prophylactic treatment,” also known as “preventive treatment,” refers to a treatment that aims at protecting a person from, or reducing the risk for a disease to which he or she has been, or may be, exposed. In some embodiments, the treatment methods described herein aim at preventing occurrence and/or recurrence of HAE.

The term “preventing” a disease in a subject refers to subjecting the subject to a pharmaceutical treatment, e.g., the administration of a drug, such that at least one symptom of the disease is prevented, that is, administered prior to clinical manifestation of the unwanted condition (e.g., disease or other unwanted state of the host animal) so that it protects the host against developing the unwanted condition. “Preventing” a disease may also be referred to as “prophylaxis” or “prophylactic treatment.”

A “prophylactically effective amount” refers to an amount effective, at dosages and for periods of time necessary, to achieve the desired prophylactic result. Typically, because a prophylactic dose is used in subjects prior to or at an earlier stage of disease, the prophylactically effective amount will be less than the therapeutically effective amount.

Antibodies Binding to Plasma Kallikrein (pKal)

Plasma kallikrein binding antibodies (anti-pKal antibodies) for use in the methods described herein can be full-length (e.g., an IgG (including an IgG1, IgG2, IgG3, IgG4), IgM, IgA (including, IgA1, IgA2), IgD, and IgE) or can include only an antigen-binding fragment (e.g., a Fab, F(ab′)₂ or scFv fragment. The binding antibody can include two heavy chain immunoglobulins and two light chain immunoglobulins, or can be a single chain antibody. Plasma kallikrein binding antibodies can be recombinant proteins such as humanized, CDR grafted, chimeric, deimmunized, or in vitro generated antibodies, and may optionally include constant regions derived from human germline immunoglobulin sequences. In one embodiment, the plasma kallikrein binding antibody is a monoclonal antibody.

In one aspect, the disclosure features an antibody (e.g., an isolated antibody) that binds to plasma kallikrein (e.g., human plasma kallikrein and/or murine kallikrein) and includes at least one immunoglobulin variable region. For example, the antibody includes a heavy chain (HC) immunoglobulin variable domain sequence and/or a light chain (LC) immunoglobulin variable domain sequence. In one embodiment, the antibody binds to and inhibits plasma kallikrein, e.g., human plasma kallikrein and/or murine kallikrein.

In some embodiments, the antibodies described herein have the same CDR sequences as DX-2930, e.g., heavy chain CDR sequences set forth as SEQ ID NOs: 5-7 and light chain CDR sequences set forth as SEQ ID NOs: 8-10. In some embodiments, the antibody comprises the same CDR sequences as DX-2930 and a LC immunoglobulin variable domain sequence that is at least 85, 88, 89, 90, 91, 92, 93, 94, 95, 96, 97, 98, 99, or 100% identical to a LC variable domain described herein (e.g., overall or in framework regions). In some embodiments, the antibody comprises the same CDR sequences as DX-2930 and an HC immunoglobulin variable domain sequence that is at least 85, 88, 89, 90, 91, 92, 93, 94, 95, 96, 97, 98, 99, or 100% identical to a HC variable domain described herein (e.g., overall or in framework regions). In some embodiments, the antibody comprises the same CDR sequences as DX-2930 and LC sequence that is at least 85, 88, 89, 90, 91, 92, 93, 94, 95, 96, 97, 98, 99, or 100% identical to a LC sequence described herein (e.g., overall or in framework regions). In some embodiments, the antibody comprises the same CDR sequences as DX-2930 and HC sequence that is at least 85, 88, 89, 90, 91, 92, 93, 94, 95, 96, 97, 98, 99, or 100% identical to a HC sequence described herein (e.g., overall or in framework regions).

The plasma kallikrein binding protein may be an isolated antibody (e.g., at least 70, 80, 90, 95, or 99% free of other proteins). In some embodiments, the plasma kallikrein binding antibody, or composition thereof, is isolated from antibody cleavage fragments (e.g., DX-2930) that are inactive or partially active (e.g., bind plasma kallikrein with a K_{i, app} of 5000 nM or greater) compared to the plasma kallikrein binding antibody. For example, the plasma kallikrein binding antibody is at least 70% free of such antibody cleavage fragments; in other embodiments the binding antibody is at least 80%, at least 90%, at least 95%, at least 99% or even 100% free from antibody cleavage fragments that are inactive or partially active.

The plasma kallikrein binding antibody may additionally inhibit plasma kallikrein, e.g., human plasma kallikrein.

In some embodiments, the plasma kallikrein binding antibody does not bind prekallikrein (e.g., human prekallikrein and/or murine prekallikrein), but binds to the active form of plasma kallikrein (e.g., human plasma kallikrein and/or murine kallikrein).

In certain embodiments, the antibody binds at or near the active site of the catalytic domain of plasma kallikrein, or a fragment thereof, or binds an epitope that overlaps with the active site of plasma kallikrein.

The antibody can bind to plasma kallikrein, e.g., human plasma kallikrein, with a binding affinity of at least 10⁵, 10⁶, 10⁷, 10⁸, 10⁹, 10¹⁰ and 10¹¹ M⁻¹. In one embodiment, the antibody binds to human plasma kallikrein with a K_{off} slower than 1×10⁻³, 5×10⁻⁴ s⁻¹, or 1×10⁻⁴ s⁻¹. In one embodiment, the antibody binds to human plasma kallikrein with a Kₒₙ faster than 1×10², 1×10³, or 5×10³ M⁻¹s⁻¹. In one embodiment, the antibody binds to plasma kallikrein, but does not bind to tissue kallikrein and/or plasma prekallikrein (e.g., the antibody binds to tissue kallikrein and/or plasma prekallikrein less effectively (e.g., 5-, 10-, 50-, 100-, or 1000-fold less or not at all, e.g., as compared to a negative control) than it binds to plasma kallikrein.

In one embodiment, the antibody inhibits human plasma kallikrein activity, e.g., with a Ki of less than 10⁻⁵, 10⁻⁶, 10⁻⁷, 10⁻⁸, 10⁻⁹, and 10⁻¹⁰ M. The antibody can have, for example, an IC₅₀ of less than 100 nM, 10 nM, 1, 0.5, or 0.2 nM. For example, the antibody may modulate plasma kallikrein activity, as well as the production of Factor XIIa (e.g., from Factor XII) and/or bradykinin (e.g., from high-molecular-weight kininogen (HMWK)). The antibody may inhibit plasma kallikrein activity, and/or the production of Factor XIIa (e.g., from Factor XII) and/or bradykinin (e.g., from high-molecular-weight kininogen (HMWK)). The affinity of the antibody for human plasma kallikrein can be characterized by a K_{D} of less than 100 nm, less than 10 nM, less than 5 nM, less than 1 nM, less than 0.5 nM. In one embodiment, the antibody inhibits plasma kallikrein, but does not inhibit tissue kallikrein (e.g., the antibody inhibits tissue kallikrein less effectively (e.g., 5-, 10-, 50-, 100-, or 1000-fold less or not at all, e.g., as compared to a negative control) than it inhibits plasma kallikrein.

In some embodiments, the antibody has an apparent inhibition constant (K_{i,app}) of less than 1000, 500, 100, 5, 1, 0.5 or 0.2 nM.

Plasma kallikrein binding antibodies may have their HC and LC variable domain sequences included in a single polypeptide (e.g., scFv), or on different polypeptides (e.g., IgG or Fab).

In one embodiment, the HC and LC variable domain sequences are components of the same polypeptide chain. In another, the HC and LC variable domain sequences are components of different polypeptide chains. For example, the antibody is an IgG, e.g., IgG1, IgG2, IgG3, or IgG4. The antibody can be a soluble Fab. In other implementations the antibody includes a Fab2′, scFv, minibody, scFv::Fc fusion, Fab::HSA fusion, HSA::Fab fusion, Fab::HSA::Fab fusion, or other molecule that comprises the antigen combining site of one of the binding proteins herein. The VH and VL regions of these Fabs can be provided as IgG, Fab, Fab2, Fab2′, scFv, PEGylated Fab, PEGylated scFv, PEGylated Fab2, VH::CH1::HSA+LC, HSA::VH::CH1+LC, LC::HSA+VH::CH1, HSA::LC+VH::CH1, or other appropriate construction.

In one embodiment, the antibody is a human or humanized antibody or is non-immunogenic in a human. For example, the antibody includes one or more human antibody framework regions, e.g., all human framework regions, or framework regions at least 85, 88, 89, 90, 91, 92, 93, 94, 95, 96, 97, 98, 99% identical to human framework regions. In one embodiment, the antibody includes a human Fc domain, or an Fc domain that is at least 95, 96, 97, 98, or 99% identical to a human Fc domain.

In one embodiment, the antibody is a primate or primatized antibody or is non-immunogenic in a human. For example, the antibody includes one or more primate antibody framework regions, e.g., all primate framework regions, or framework regions at least 85, 88, 89, 90, 91, 92, 93, 94, 95, 96, 97, 98, 99% identical to primate framework regions. In one embodiment, the antibody includes a primate Fc domain, or an Fc domain that is at least 95, 96, 97, 98, or 99% identical to a primate Fc domain. “Primate” includes humans (Homo sapiens), chimpanzees (Pan troglodytes and Pan paniscus (bonobos)), gorillas (Gorilla gorilla), gibbons, monkeys, lemurs, aye-ayes (Daubentonia madagascariensis), and tarsiers. In some embodiments, the affinity of the primate antibody for human plasma kallikrein is characterized by a K_{D} of less than 1000, 500, 100, 10, 5, 1, 0.5 nM, e.g., less than 10 nM, less than 1 nM, or less than 0.5 nM.

In certain embodiments, the antibody includes no sequences from mice or rabbits (e.g., is not a murine or rabbit antibody).

In some embodiments, the antibody used in the methods described herein may be DX-2930 as described herein or a functional variant thereof.

In one example, a functional variant of DX-2930 comprises the same complementary determining regions (CDRs) as DX-2930. In another example, the functional variants of DX-2930 may contain one or more mutations (e.g., conservative substitutions) in the FRs of either the V_{H} or the V_{L} as compared to those in the V_{H} and V_{L} of DX-2930. Preferably, such mutations do not occur at residues which are predicted to interact with one or more of the CDRs, which can be determined by routine technology. In other embodiments, the functional variants described herein contain one or more mutations (e.g., 1, 2, or 3) within one or more of the CDR regions of DX-2930. Preferably, such functional variants retain the same regions/residues responsible for antigen-binding as the parent. In yet other embodiments, a functional variant of DX-2930 may comprise a V_{H} chain that comprises an amino acid sequence at least 85% (e.g., 90%, 92%, 94%, 95%, 96%, 97%, 98%, or 99%) identical to that of the V_{H} of DX-2930 and/or a V_{L} chain that has an amino acid sequence at least 85% (e.g., 90%, 92%, 94%, 95%, 96%, 97%, 98%, or 99%) identical to that of the V_{L} of DX-2930. These variants are capable of binding to the active form of plasma kallikrein and preferably do not bind to prekallikrein.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the antibody used in the methods and compositions described herein may be the DX-2930 antibody. The heavy and light chain full and variable sequences for DX-2930 are provided below, with signal sequences in italics. The CDRs are boldfaced and underlined.

DX-2930 Heavy Chain Amino Acid Sequence (451 amino
acids, 49439.02 Da)
(SEQ ID NO: 1)
MGWSCILFLVATATGAHSEVQLLESGGGLVQPGGSLRLSCAASGFTFSHYI
MMWVRQAPGKGLEWVSGIYSSGGITVYADSVKGRFTISRDNSKNTLYLQMN
SLRAEDTAVYYCAYRRIGVPRRDEFDIWGQGTMVTVSSASTKGPSVFPLAP
SSKSTSGGTAALGCLVKDYFPEPVTVSWNSGALTSGVHTFPAVLQSSGLYS
LSSVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHY
TQKSLSLSPG
DX-2930 Light Chain Amino Acid Sequence (213 amino
acids, 23419.08 Da)
(SEQ ID NO: 2)
MGWSCILFLVATATGAHSDIQMTQSPSTLSASVGDRVTITCRASQSISSWL
AWYQQKPGKAPKLLIYKASTLESGVPSRFSGSGSGTEFTLTISSLQPDDFA
TYYCQQYNTYWTFGQGTKVEIKRTVAAPSVFIFPPSDEQLKSGTASVVCLL
NKHKVYACEVTHQGLSSPVTKSFNRNFYPREAKVQWKVDNALQSGNSQESV
TEQDSKDSTYSLSSTLTLSKADYEGEC
DX-2930 Heavy Chain Variable Domain Amino Acid
Sequence
(SEQ ID NO: 3)
EVQLLESGGGLVQPGGSLRLSCAASGFTFSHYIMMWVRQAPGKGLEWVSGI
YSSGGITVYADSVKGRFTISRDNSKNTLYLQMNSLRAEDTAVYYCAYRRIG
VPRRDEFDIWGQGTMVTVSS
DX-2930 Light Chain Variable Domain Amino Acid
Sequence
(SEQ ID NO: 4)
DIQMTQSPSTLSASVGDRVTITCRASQSISSWLAWYQQKPGKAPKWIYKAS
TLESGVPSRFSGSGSGTEFTLTISSLQPDDFATYYCQQYNTYWTFGQGTKV
EIK

TABLE 1
CDRs for DX-2930.

| CDR | Amino acid sequence |
|---|---|
| Heavy chain CDR1 | HYIMM (SEQ ID NO: 5) |
| Heavy chain CDR2 | GIYSSGGITVYADSVKG (SEQ ID NO: 6) |
| Heavy chain CDR3 | RRIGVPRRDEFDI (SEQ ID NO: 7) |
| Light chain CDR1 | RASQSISSWLA (SEQ ID NO: 8) |
| Light chain CDR2 | KASTLES (SEQ ID NO: 9) |
| Light chain CDR3 | QQYNTYWT (SEQ ID NO: 10) |

### Antibody Preparation

An antibody as described herein (e.g., DX-2930) can be made by any method known in the art. See, for example, Harlow and Lane, (1988) Antibodies: A Laboratory Manual, Cold Spring Harbor Laboratory, New York and Greenfield, (2013) Antibodies: A Laboratory Manual, Second edition, Cold Spring Harbor Laboratory Press.

The sequence encoding the antibody of interest, e.g., DX-2930, may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of PKal. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)₂ fragments can be produced by pepsin digestion of an antibody molecule, and Fab fragments that can be generated by reducing the disulfide bridges of F(ab′)₂ fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated or synthesized. The DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a PKal can be identified from the library following routine procedures. Positive clones can be subjected to further screening to identify those that inhibits PKal activity.

Some antibodies, e.g., Fabs, can be produced in bacterial cells, e.g., E. coli cells (see e.g., Nadkarni, A. et al., 2007 Protein Expr Purif 52(1):219-29). For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

Antibodies can also be produced in eukaryotic cells. In one embodiment, the antibodies (e.g., scFv's) are expressed in a yeast cell such as Pichia (see, e.g., Powers et al., 2001, J. Immunol. Methods. 251:123-35; Schoonooghe S. et al., 2009 BMC Biotechnol. 9:70; Abdel-Salam, H A. et al., 2001 Appl Microbiol Biotechnol 56(1-2):157-64; Takahashi K. et al., 2000 Biosci Biotechnol Biochem 64(10):2138-44; Edqvist, J. et al., 1991 J Biotechnol 20(3):291-300), Hanseula, or Saccharomyces. One of skill in the art can optimize antibody production in yeast by optimizing, for example, oxygen conditions (see e.g., Baumann K., et al. 2010 BMC Syst. Biol. 4:141), osmolarity (see e.g., Dragosits, M. et al., 2010 BMC Genomics 11:207), temperature (see e.g., Dragosits, M. et al., 2009 J Proteome Res. 8(3):1380-92), fermentation conditions (see e.g., Ning, D. et al. 2005 J. Biochem. and Mol. Biol. 38(3): 294-299), strain of yeast (see e.g., Kozyr, A V et al. 2004 Mol Biol (Mosk) 38(6):1067-75; Horwitz, A H. et al., 1988 Proc Natl Acad Sci USA 85(22):8678-82; Bowdish, K. et al. 1991 J Biol Chem 266(18):11901-8), overexpression of proteins to enhance antibody production (see e.g., Gasser, B. et al., 2006 Biotechol. Bioeng. 94(2):353-61), level of acidity of the culture (see e.g., Kobayashi H., et al., 1997 FEMS Microbiol Lett 152(2):235-42), concentrations of substrates and/or ions (see e.g., Ko J H. et al., 2996 Appl Biochem Biotechnol 60(1):41-8). In addition, yeast systems can be used to produce antibodies with an extended half-life (see e.g., Smith, B J. et al. 2001 Bioconjug Chem 12(5):750-756).

In one preferred embodiment, antibodies are produced in mammalian cells. Preferred mammalian host cells for expressing the clone antibodies or antigen-binding fragments thereof include Chinese Hamster Ovary (CHO cells) (including dhfr− CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601 621), lymphocytic cell lines, e.g., NS0 myeloma cells and SP2 cells, COS cells, HEK293T cells (J. Immunol. Methods (2004) 289(1-2):65-80), and a cell from a transgenic animal, e.g., a transgenic mammal. For example, the cell is a mammary epithelial cell.

In some embodiments, plasma kallikrein binding antibodies are produced in a plant or cell-free based system (see e.g., Galeffi, P., et al., 2006 J Transl Med 4:39).

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

In an exemplary system for recombinant expression of an antibody, or antigen-binding portion thereof, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G coupled matrix.

For antibodies that include an Fc domain, the antibody production system may produce antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof, 1992, Adv. Immunol. 51:1-84; Jefferis et al., 1998, Immunol. Rev. 163:59-76). In one embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

### Pharmaceutical Compositions

An antibody as described herein (e.g., DX-2930) can be present in a composition, e.g., a pharmaceutically acceptable composition or pharmaceutical composition. The antibody as described herein (e.g., DX-2930) can be formulated together with a pharmaceutically acceptable carrier. In some embodiments, 150 mg or 300 mg of DX-2930 antibody are present in a composition optionally with a pharmaceutically acceptable carrier, e.g., a pharmaceutically acceptable composition or pharmaceutical composition.

A pharmaceutically acceptable carrier includes any and all solvents, dispersion media, coatings, antibacterial and antifungal agents, isotonic and absorption delaying agents, and the like that are physiologically compatible. Preferably, the carrier is suitable for subcutaneous, intravenous, intramuscular, parenteral, spinal, or epidermal administration (e.g., by injection or infusion), although carriers suitable for inhalation and intranasal administration are also contemplated.

The pharmaceutically acceptable carrier in the pharmaceutical composition described herein may include one or more of a buffering agent, an amino acid, and a tonicity modifier. Any suitable buffering agent or combination of buffering agents may be used in the pharmaceutical composition described herein to maintain or aid in maintaining an appropriate pH of the composition. Non-limiting examples of buffering agents include sodium phosphate, potassium phosphate, citric acid, sodium succinate, histidine, Tris, and sodium acetate. In some embodiments, the buffering agents may be at a concentration of about 5-100 mM, 5-50 mM, 10-50 mM, 15-50 mM, or about 15-40 mM. For example, the one or more buffering agents may be at a concentration of about 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 35 mM, 36 mM, 37 mM, 38 mM, 39 mM, or about 40 mM. In some examples, the pharmaceutically acceptable carrier comprises sodium phosphate and citric acid, which may be at a concentration of about 30 mM and about 19 mM, respectively.

In some embodiments, the pharmaceutically acceptable carrier includes one or more amino acids, which may decrease aggregation of the antibody and/or increase stability of the antibody during storage prior to administration. Exemplary amino acids for use in making the pharmaceutical compositions described herein include, but are not limited to, alanine, arginine, asparagine, aspartic acid, glycine, histidine, lysine, proline, or serine. In some examples, the concentration of the amino acid in the pharmaceutical composition may be about 5-100 mM, 10-90 mM, 20-80 mM, 30-70 mM, 40-60 mM, or about 45-55 mM. In some examples, the concentration of the amino acid (e.g., histidine) may be about 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, or about 60 mM. In one example, the pharmaceutical composition contains histidine at a concentration of about 50 mM.

Any suitable tonicity modifier may be used for preparing the pharmaceutical compositions described herein. In some embodiments, the tonicity modifier is a salt or an amino acid. Examples of suitable salts include, without limitation, sodium chloride, sodium succinate, sodium sulfate, potassium chloride, magnesium chloride, magnesium sulfate, and calcium chloride. In some embodiments, the tonicity modifier in the pharmaceutical composition may be at a concentration of about 10-150 mM, 50-150 mM, 50-100 mM, 75-100 mM, or about 85-95 mM. In some embodiments, the tonicity modifier may be at a concentration of about 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or about 100 mM. In one example, the tonicity modifier may be sodium chloride, which may be at a concentration of about 90 mM.

The pharmaceutically acceptable carrier in the pharmaceutical compositions described herein may further comprise one or more pharmaceutically acceptable excipients. In general, pharmaceutically acceptable excipients are pharmacologically inactive substances. Non-limiting examples of excipients include lactose, glycerol, xylitol, sorbitol, mannitol, maltose, inositol, trehalose, glucose, bovine serum albumin (BSA), dextran, polyvinyl acetate (PVA), hydroxypropyl methylcellulose (HPMC), polyethyleneimine (PEI), gelatin, polyvinylpyrrolidone (PVP), hydroxyethylcellulose (HEC), polyethylene glycol (PEG), ethylene glycol, glycerol, dimethysulfoxide (DMSO), dimethylformamide (DMF), polyoxyethylene sorbitan monolaurate (Tween-20), polyoxyethylene sorbitan monooleate (Tween-80), sodium dodecyl sulphate (SDS), polysorbate, polyoxyethylene copolymer, potassium phosphate, sodium acetate, ammonium sulfate, magnesium sulfate, sodium sulfate, trimethylamine N-oxide, betaine, zinc ions, copper ions, calcium ions, manganese ions, magnesium ions, CHAPS, sucrose monolaurate and 2-O-beta-mannoglycerate. In some embodiments, the pharmaceutically acceptable carrier comprises an excipient between about 0.001%-0.1%, 0.001%-0.05%, 0.005-0.1%, 0.005%-0.05%, 0.008%-0.05%, 0.008%-0.03% or about 0.009%-0.02%. In some embodiments, the excipient is at about 0.005%, 0.006%, 0.007%, 0.008%, 0.009%, 0.01%, 0.02%, 0.03%, 0.04%, 0.05%, 0.06%, 0.07%, 0.08%, 0.09%, or about 0.1%. In some embodiments, the excipient is polyoxyethylene sorbitan monooleate (Tween-80). In one example, the pharmaceutically acceptable carrier contains 0.01% Tween-80.

In some examples, the pharmaceutical composition described herein comprises the anti-pKal antibody as also described herein (e.g., DX-2930), and one or more of sodium phosphate (e.g., sodium phosphate dibasic dihydrate), citric acid (e.g., citric acid monohydrate), histidine (e.g., L-histidine), sodium chloride, and Polysorbate 80. For example, the pharmaceutical composition may comprise the antibody, sodium phosphate, citric acid, histidine, sodium chloride, and Polysorbate 80. In some examples, the antibody is formulated in about 30 mM sodium phosphate, about 19 mM citric acid, about 50 mM histidine, about 90 mM sodium chloride, and about 0.01% Polysorbate 80. The concentration of the antibody (e.g., DX-2930) in the composition can be about 150 mg/mL or 300 mg/mL. In one example, the composition comprises or consists of about 150 mg DX-2930 per 1 mL solution, about 30 mM sodium phosphate dibasic dihydrate, about 19 mM (e.g., 19.6 mM) citric acid monohydrate, about 50 mM L-histidine, about 90 mM sodium chloride, and about 0.01% Polysorbate 80. In another example, the composition comprises or consists of about 300 mg DX-2930 per 1 mL solution, about 30 mM sodium phosphate dibasic dihydrate, about 19 mM (e.g., 19.6 mM) citric acid monohydrate, about 50 mM L-histidine, about 90 mM sodium chloride, and about 0.01% Polysorbate 80.

A pharmaceutically acceptable salt is a salt that retains the desired biological activity of the compound and does not impart any undesired toxicological effects (see, e.g., Berge, S. M., et al., 1977, J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like.

The compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Many compositions are in the form of injectable or infusible solutions, such as compositions similar to those used for administration of humans with antibodies. An exemplary mode of administration is parenteral (e.g., intravenous, subcutaneous, intraperitoneal, intramuscular). In one embodiment, the plasma kallikrein binding protein is administered by intravenous infusion or injection. In another embodiment, the plasma kallikrein binding protein is administered by intramuscular injection. In another embodiment, the plasma kallikrein binding protein is administered by subcutaneous injection. In another preferred embodiment, the plasma kallikrein binding protein is administered by intraperitoneal injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In some embodiments, the antibody is administered subcutaneously.

The composition can be formulated as a solution, microemulsion, dispersion, liposome, or other ordered structure suitable to high drug concentration. Sterile injectable solutions can be prepared by incorporating the binding protein in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An antibody as described herein (e.g., DX-2930) can be administered by a variety of methods, including intravenous injection, subcutaneous injection, or infusion. For example, for some therapeutic applications, the antibody can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are available. See, e.g., Sustained and Controlled Release Drug Delivery Systems, J. R. Robinson, ed., 1978, Marcel Dekker, Inc., New York.

Pharmaceutical compositions can be administered with medical devices. For example, in one embodiment, a pharmaceutical composition disclosed herein can be administered with a device, e.g., a needleless hypodermic injection device, a pump, or implant.

In certain embodiments, an antibody as described herein (e.g., DX-2930) can be formulated to ensure proper distribution in vivo. For example, the blood-brain barrier (BBB) excludes many highly hydrophilic compounds. To ensure that the therapeutic compounds disclosed herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade, 1989, J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms can be dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody as described herein (e.g., DX-2930) is about 150 mg or 300 mg. As will be understood by one of ordinary skill in the art, a therapeutically or prophylactically effective amount of an antibody may be lower for a pediatric subject than for an adult subject. In some embodiments, the effective amount that is administered to a pediatric subject is a fixed dose or a weight based dose. In some embodiments, effective amount that is less than about 150 mg or 300 mg is administered to a pediatric subject. In some embodiments, a therapeutically or prophylactically effective amount of an antibody is administered every two weeks or every four weeks for a first treatment period. In some embodiments, the antibody may be administered to the subject for a second treatment period. In some embodiments, the therapeutically or prophylactically effective amount of the antibody in the first treatment period is different than the therapeutically or prophylactically effective amount of the antibody in the second treatment period. In some embodiments, the therapeutically or prophylactically effective amount of the antibody in the first treatment period is 150 mg and the therapeutically or prophylactically effective amount of the antibody in the second treatment period is 300 mg. In some embodiments, the therapeutically or prophylactically effective amount of the antibody in the first treatment period is the same as the therapeutically or prophylactically effective amount of the antibody in the second treatment period. In one example, therapeutically or prophylactically effective amount of the antibody in the first treatment period and the second treatment period is 300 mg.

In some embodiments, an exemplary, non-limiting range for a therapeutically or prophylactically effective amount of an antibody as described herein (e.g., DX-2930) is about 300 mg. In some embodiments, a therapeutically or prophylactically effective amount of an antibody is administered in a single dose. If the subject experiences a HAE attack, the antibody may be further administered to the subject in multiple doses, such in doses of about 300 mg administered every two weeks.

### Kits

An antibody as described herein (e.g., DX-2930) can be provided in a kit, e.g., as a component of a kit. For example, the kit includes (a) a DX-2930 antibody, e.g., a composition (e.g., a pharmaceutical composition) that includes the antibody, and, optionally (b) informational material. The informational material can be descriptive, instructional, marketing or other material that relates to a method described herein and/or the use of an antibody as described herein (e.g., DX-2930), e.g., for a method described herein. In some embodiments, the kit comprises one or more doses of DX-2930. In some embodiments, the one or more doses are 150 mg or 300 mg.

The informational material of the kit is not limited in its form. In one embodiment, the informational material can include information about production of the compound, molecular weight of the compound, concentration, date of expiration, batch or production site information, and so forth. In one embodiment, the informational material relates to using the antibody to treat, prevent, or diagnosis of disorders and conditions, e.g., a plasma kallikrein associated disease or condition.

In one embodiment, the informational material can include instructions to administer an antibody as described herein (e.g., DX-2930) in a suitable manner to perform the methods described herein, e.g., in a suitable dose, dosage form, mode of administration or dosing schedule (e.g., a dose, dosage form, dosing schedule or mode of administration described herein). In another embodiment, the informational material can include instructions to administer an antibody as described herein (e.g., DX-2930) to a suitable subject, e.g., a human, e.g., a human having, or at risk for, a plasma kallikrein associated disease or condition. For example, the material can include instructions to administer an antibody as described herein (e.g., DX-2930) to a patient with a disorder or condition described herein, e.g., a plasma kallikrein associated disease, e.g., according to a dosing schedule described herein. The informational material of the kits is not limited in its form. In many cases, the informational material, e.g., instructions, is provided in print but may also be in other formats, such as computer readable material.

An antibody as described herein (e.g., DX-2930) can be provided in any form, e.g., liquid, dried or lyophilized form. It is preferred that an antibody be substantially pure and/or sterile. When an antibody is provided in a liquid solution, the liquid solution preferably is an aqueous solution, with a sterile aqueous solution being preferred. When an antibody is provided as a dried form, reconstitution generally is by the addition of a suitable solvent. The solvent, e.g., sterile water or buffer, can optionally be provided in the kit.

The kit can include one or more containers for the composition containing an antibody as described herein (e.g., DX-2930). In some embodiments, the kit contains separate containers, dividers or compartments for the composition and informational material. For example, the composition can be contained in a bottle, vial, or syringe, and the informational material can be contained in association with the container. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an antibody as described herein (e.g., DX-2930). For example, the kit includes a plurality of syringes, ampules, foil packets, or blister packs, each containing a single unit dose of an antibody as described herein (e.g., DX-2930). The containers of the kits can be air tight, waterproof (e.g., impermeable to changes in moisture or evaporation), and/or light-tight.

The kit optionally includes a device suitable for administration of the composition, e.g., a syringe, or any such delivery device. In one embodiment, the device is an implantable device that dispenses metered doses of the antibody. The disclosure also features a method of providing a kit, e.g., by combining components described herein.

### Treatment

In some aspects, the disclosure provides the use of an antibody as described herein (e.g., DX-2930) in treating HAE.

(i) Hereditary Angioedema

Hereditary angioedema (HAE) is also known as “Quincke edema,” C1 esterase inhibitor deficiency, C1 inhibitor deficiency, and hereditary angioneurotic edema (HANE). HAE is characterized by unpredictable, recurrent attacks of severe subcutaneous or submucosal swelling (angioedema), which can affect, e.g., the limbs, face, genitals, gastrointestinal tract, and airway (Zuraw, 2008). Symptoms of HAE include, e.g., swelling in the arms, legs, lips, eyes, tongue, and/or throat; airway blockage that can involve throat (larynx) swelling, sudden hoarseness and/or cause death from asphyxiation (Bork et al., 2012; Bork et al., 2000). Approximately 50% of all HAE patients will experience a laryngeal attack in their lifetime, and there is no way to predict which patients are at risk of a laryngeal attack (Bork et al., 2003; Bork et al., 2006). HAE symptoms also include repeat episodes of abdominal cramping without obvious cause; and/or swelling of the intestines, which can be severe and can lead to abdominal cramping, vomiting, dehydration, diarrhea, pain, shock, and/or intestinal symptoms resembling abdominal emergencies, which may lead to unnecessary surgery (Zuraw, 2008). Swelling may last up to five or more days. About one-third of individuals with this HAE develop a non-itchy rash called erythema marginatum during an attack. Most patients suffer multiple attacks per year.

HAE is an orphan disorder, the exact prevalence of which is unknown, but current estimates range from 1 per 10,000 to 1 per 150,000 persons, with many authors agreeing that 1 per 50,000 is likely the closest estimate (Bygum, 2009; Goring et al., 1998; Lei et al., 2011; Nordenfelt et al., 2014; Roche et al., 2005).

Plasma kallikrein plays a critical role in the pathogenesis of HAE attacks (Davis, 2006; Kaplan and Joseph, 2010). In normal physiology, C1-INH regulates the activity of plasma kallikrein as well as a variety of other proteases, such as C1r, C1s, factor XIa, and factor XIIa. Plasma kallikrein regulates the release of bradykinin from high molecular weight kininogen (HMWK). Due to a deficiency of C1-INH in HAE, uncontrolled plasma kallikrein activity occurs and leads to the excessive generation of bradykinin. Bradykinin is a vasodilator which is thought to be responsible for the characteristic HAE symptoms of localized swelling, inflammation, and pain (Craig et al., 2012; Zuraw et al., 2013).

Swelling of the airway can be life threatening and causes death in some patients. Mortality rates are estimated at 15-33%. HAE leads to about 15,000-30,000 emergency department visits per year.

Trauma or stress, e.g., dental procedures, sickness (e.g., viral illnesses such as colds and the flu), menstruation, and surgery can trigger an attack of angioedema. To prevent acute attacks of HAE, patients can attempt to avoid specific stimuli that have previously caused attacks. However, in many cases, an attack occurs without a known trigger. Typically, HAE symptoms first appear in childhood and worsen during puberty. On average, untreated individuals have an attack every 1 to 2 weeks, and most episodes last for about 3 to 4 days (ghr.nlm.nih.gov/condition/hereditary-angioedema). The frequency and duration of attacks vary greatly among people with hereditary angioedema, even among people in the same family.

There are three types of HAE, known as types I, II, and III, all of which can be treated by the methods described herein. It is estimated that HAE affects 1 in 50,000 people, that type I accounts for about 85 percent of cases, type II accounts for about 15 percent of cases, and type III is very rare. Type III is the most newly described form and was originally thought to occur only in women, but families with affected males have been identified.

HAE is inherited in an autosomal dominant pattern, such that an affected person can inherit the mutation from one affected parent. New mutations in the gene can also occur, and thus HAE can also occur in people with no history of the disorder in their family. It is estimated that 20-25% of cases result from a new spontaneous mutation.

Mutations in the SERPING1 gene cause hereditary angioedema type I and type II. The SERPING1 gene provides instructions for making the C1 inhibitor protein, which is important for controlling inflammation. C1 inhibitor blocks the activity of certain proteins that promote inflammation. Mutations that cause hereditary angioedema type I lead to reduced levels of C1 inhibitor in the blood. In contrast, mutations that cause type II result in the production of a C1 inhibitor that functions abnormally. Approximately 85% of patients have Type I HAE, characterized by very low production of functionally normal C1-INH protein, while the remaining approximately 15% of patients have Type II HAE and produce normal or elevated levels of a functionally impaired C1-INH (Zuraw, 2008). Without the proper levels of functional C1 inhibitor, excessive amounts of bradykinin are generated from high molecular weight kininogen (HMWK), and there is increased vascular leakage mediated by bradykinin binding to the B2 receptor (B2-R) on the surface of endothelial cells (Zuraw, 2008). Bradykinin promotes inflammation by increasing the leakage of fluid through the walls of blood vessels into body tissues. Excessive accumulation of fluids in body tissues causes the episodes of swelling seen in individuals with hereditary angioedema type I and type II.

Mutations in the F12 gene are associated with some cases of hereditary angioedema type III. The F12 gene provides instructions for making coagulation factor XII. In addition to playing a critical role in blood clotting (coagulation), factor XII is also an important stimulator of inflammation and is involved in the production of bradykinin. Certain mutations in the F12 gene result in the production of factor XII with increased activity. As a result, more bradykinin is generated and blood vessel walls become more leaky, which leads to episodes of swelling. The cause of other cases of hereditary angioedema type III remains unknown. Mutations in one or more as-yet unidentified genes may be responsible for the disorder in these cases.

HAE can present similarly to other forms of angioedema resulting from allergies or other medical conditions, but it differs significantly in cause and treatment. When hereditary angioedema is misdiagnosed as an allergy, it is most commonly treated with antihistamines, steroids, and/or epinephrine, which are typically ineffective in HAE, although epinephrine can be used for life-threatening reactions. Misdiagnoses have also resulted in unnecessary exploratory surgery for patients with abdominal swelling, and in some HAE patients abdominal pain has been incorrectly diagnosed as psychosomatic.

Like adults, children with HAE can suffer from recurrent and debilitating attacks. Symptoms may present very early in childhood, and upper airway angioedema has been reported in HAE patients as young as the age of 3 (Bork et al., 2003). In one case study of 49 pediatric HAE patients, 23 had suffered at least one episode of airway angioedema by the age of 18 (Farkas, 2010). An important unmet medical need exists among children with HAE, especially adolescents, since the disease commonly worsens after puberty (Bennett and Craig, 2015; Zuraw, 2008).

C1 inhibitor therapies, as well as other therapies for HAE, are described in Kaplan, A. P., J Allergy Clin Immunol, 2010, 126(5):918-925.

Acute treatment of HAE attacks is provided to halt progression of the edema as quickly as possible. C1 inhibitor concentrate from donor blood, which is administered intravenously, is one acute treatment; however, this treatment is not available in many countries. In emergency situations where C1 inhibitor concentrate is not available, fresh frozen plasma (FFP) can be used as an alternative, as it also contains C1 inhibitor.

Purified C1 inhibitor, derived from human blood, has been used in Europe since 1979. Several C1 inhibitor treatments are now available in the U.S. and two C1 inhibitor products are now available in Canada. Berinert P (CSL Behring), which is pasteurized, was approved by the F.D.A. in 2009 for acute attacks. Cinryze (ViroPharma), which is nanofiltered, was approved by the F.D.A. in 2008 for prophylaxis. Rhucin (Pharming) is a recombinant C1 inhibitor under development that does not carry the risk of infectious disease transmission due to human blood-borne pathogens.

Treatment of an acute HAE attack also can include medications for pain relief and/or IV fluids.

Other treatment modalities can stimulate the synthesis of C1 inhibitor, or reduce C1 inhibitor consumption. Androgen medications, such as danazol, can reduce the frequency and severity of attacks by stimulating production of C1 inhibitor.

Helicobacter pylori can trigger abdominal attacks. Antibiotics to treat H. pylori will decrease abdominal attacks.

Newer treatments attack the contact cascade. Ecallantide (KALBITOR®, DX-88, Dyax) inhibits plasma kallikrein and has been approved in the U.S. Icatibant (FIRAZYR®, Shire) inhibits the bradykinin B2 receptor, and has been approved in Europe and the U.S.

Diagnosis of HAE can rely on, e.g., family history and/or blood tests. Laboratory findings associated with HAE types I, II, and III are described, e.g., in Kaplan, A. P., J Allergy Clin Immunol, 2010, 126(5):918-925. In type I HAE, the level of C1 inhibitor is decreased, as is the level of C4, whereas C1q level is normal. In type II HAE, the level of C1 inhibitor is normal or increased; however, C1 inhibitor function is abnormal. C4 level is decreased and C1q level is normal. In type III, the levels of C1 inhibitor, C4, and C1q can all be normal.

Symptoms of HAE can be assessed, for example, using questionnaires, e.g., questionnaires that are completed by patients, clinicians, or family members. Such questionnaires are known in the art and include, for example, visual analog scales. See, e.g., McMillan, C. V. et al. Patient. 2012; 5(2):113-26. In some embodiments, the subject has HAE type I or HAE type II. HAE type I or HAE type II may be diagnosed using any method known in the art, such as by clinical history consistent with HAE (e.g., subcutaneous or mucosal, nonpruritic swelling episodes) or diagnostic testing (e.g., C1-INH functional testing and C4 level assessment).

(ii) Treating HAE with Anti-PKal Antibodies

The disclosure provides methods of treating (e.g., ameliorating, stabilizing, or eliminating one or more symptoms) of hereditary angioedema (HAE) by administering an antibody described herein (e.g., a therapeutically effective amount of an antibody described herein) to a subject having or suspected of having HAE, e.g., according to a dosing schedule described herein. Additionally provided are methods of treating HAE by administering an antibody described herein (e.g., a therapeutically effective amount of an antibody described herein), e.g., according to a dosing schedule described herein, or in combination with a second therapy, e.g., with one other agent, e.g., described herein. The disclosure also provides methods of preventing HAE or a symptom thereof by administering an antibody described herein (e.g., a prophylactically effective amount of an antibody described herein) to a subject at risk of developing HAE (e.g., a subject having a family member with HAE or a genetic predisposition thereto), e.g., according to a dosing schedule described herein. In some examples, the subject may be a human patient who has no HAE symptoms at the time of the treatment. In some embodiments, the subject is a human patient that has HAE type I or HAE type II. In some embodiments, the subject is a human patient that has experienced at least two (e.g., 2, 3, 4, 5 or more) HAE attacks in the year prior to the treatment.

In some embodiments, the subject is female. In some embodiments, the subject is a pediatric subject. In some embodiments, the subject is an adolescent less than 18 years old. In some embodiments, the subject is an adolescent between the ages of 12 and 18 years old. In some embodiments, the subject is between the ages of 40 and less than 65 years old.

In some embodiments, the subject may be defined by gender. For example, in some embodiments, the subject is female.

In some embodiments, the human subject is defined by weight. In some embodiments, the human subject weighs less than 50 kg. In some embodiments, the human subject weighs between 50 kg and 75 kg. In some embodiments the human subject weighs between 75 kg and 100 kg. In some embodiments, the human subject weighs 100 kg or more.

In some embodiments, any of the human patient subgroups may be given the anti-pKal antibody (e.g., DX-2930) at about 300 mg every two weeks. In other instances, such a human patient may be given the antibody at about 150 mg every two or four weeks. In yet other instances, such a human patient may be given the antibody at about 300 mg every four weeks.

In some embodiments, the human subject is defined by prior history of laryngeal attacks or absence thereof. In some embodiments, the subject has experienced at least one (e.g., 1, 2, 3, 4, 5, or more) laryngeal attack (i.e. laryngeal HAE attack) prior to administration of the antibodies described herein. In some embodiments, the subject has not experienced a laryngeal attack prior to administration of the antibodies described herein.

Treating includes administering an amount effective to alleviate, relieve, alter, remedy, ameliorate, improve or affect the disorder, the symptoms of the disorder or the predisposition toward the disorder. The treatment may also delay onset, e.g., prevent onset, or prevent deterioration of a disease or condition.

Methods of administering DX-2930 antibodies are also described in “Pharmaceutical Compositions.” Suitable dosages of the antibody used can depend on the age and weight of the subject and the particular drug used. The antibody can be used as competitive agents to inhibit, reduce an undesirable interaction, e.g., between plasma kallikrein and its substrate (e.g., Factor XII or HMWK). The dose of the antibody can be the amount sufficient to block 90%, 91%, 92%, 93%, 94%, 95%, 96%, 97%, 98% 99%, or 99.9% of the activity of plasma kallikrein in the patient, especially at the site of disease. In some embodiments, 150 mg or 300 mg of the antibody is administered every two weeks or every four weeks. In some embodiments, the antibody is administered to the subject in a first treatment period comprising administration of 150 mg or 300 mg of the antibody every two weeks or every four weeks. In some embodiments, the antibody is administered to the subject in a second treatment period following the first treatment period. In some embodiments, 300 mg of the antibody is administered in a single dose. If the subject experiences an HAE attack after the single dose, the antibody may be administered at 300 mg every two weeks.

In one embodiment, the antibodies are used to inhibit an activity (e.g., inhibit at least one activity of plasma kallikrein, e.g., reduce Factor XIIa and/or bradykinin production) of plasma kallikrein, e.g., in vivo. The binding proteins can be used by themselves or conjugated to an agent, e.g., a cytotoxic drug, cytotoxin enzyme, or radioisotope.

The antibodies can be used directly in vivo to eliminate antigen-expressing cells via natural complement-dependent cytotoxicity (CDC) or antibody dependent cellular cytotoxicity (ADCC). The antibodies described herein can include complement binding effector domain, such as the Fc portions from IgG1, -2, or -3 or corresponding portions of IgM which bind complement. In one embodiment, a population of target cells is ex vivo treated with an antibody described herein and appropriate effector cells. The treatment can be supplemented by the addition of complement or serum containing complement. Further, phagocytosis of target cells coated with an antibody described herein can be improved by binding of complement proteins. In another embodiment target, cells coated with the antibody which includes a complement binding effector domain are lysed by complement.

Methods of administering DX-2930 antibodies are described in “Pharmaceutical Compositions.” Suitable dosages of the molecules used will depend on the age and weight of the subject and the particular drug used. The antibodies can be used as competitive agents to inhibit or reduce an undesirable interaction, e.g., between a natural or pathological agent and the plasma kallikrein.

A therapeutically effective amount of an antibody as described herein, can be administered to a subject having, suspected of having, or at risk for HAE, thereby treating (e.g., ameliorating or improving a symptom or feature of a disorder, slowing, stabilizing and/or halting disease progression) the disorder.

The antibody described herein can be administered in a therapeutically effective amount. A therapeutically effective amount of an antibody is the amount which is effective, upon single or multiple dose administration to a subject, in treating a subject, e.g., curing, alleviating, relieving or improving at least one symptom of a disorder in a subject to a degree beyond that expected in the absence of such treatment.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. In other examples, a bolus may be administered followed by several doses over time or the dose may be proportionally reduced or increased as indicated by the exigencies of the therapeutic situation. In other examples, a dose may be divided into several doses and be administered over time. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

In some embodiments, an antibody as described herein is administered in a dosage regimen during a first treatment period. In some embodiments, the antibody is administered in the first treatment period in multiple doses. In this period, the therapeutically or prophylactically effective amount of the antibody (e.g., DX-2930) can be about 150 mg or 300 mg and is administered every week, every two weeks, every three weeks, every four weeks, every five weeks, every six weeks, every seven weeks, every eight weeks or longer. In some embodiments, the therapeutically or prophylactically effective amount of the antibody (e.g., DX-2930) can be about 300 mg and is administered to a female subject every week, every two weeks, every three weeks, every four weeks, every five weeks, every six weeks, every seven weeks, every eight weeks or longer. In some embodiments, the therapeutically or prophylactically effective amount of the antibody (e.g., DX-2930) can be about 300 mg and is administered to a subject that is less than 18 years old every week, every two weeks, every three weeks, every four weeks, every five weeks, every six weeks, every seven weeks, every eight weeks or longer. In some embodiments, the therapeutically or prophylactically effective amount of the antibody (e.g., DX-2930) can be about 300 mg and is administered to a subject that is between the ages of 40 and 65 years old every week, every two weeks, every three weeks, every four weeks, every five weeks, every six weeks, every seven weeks, every eight weeks or longer.

In some embodiments, the therapeutically or prophylactically effective amount of the antibody (e.g., DX-2930) can be about 300 mg and is administered to a subject that is greater than or equal to 65 years old every week, every two weeks, every three weeks, every four weeks, every five weeks, every six weeks, every seven weeks, every eight weeks or longer. In specific examples, the antibody is given to the subject at about 300 mg every two weeks. In other specific examples, the antibody is given to the subject at about 300 mg every four weeks.

In some embodiments, the therapeutically or prophylactically effective amount of the antibody (e.g., DX-2930) can be about 300 mg and is administered to a subject that has experienced at least one prior laryngeal HAE attack every week, every two weeks, every three weeks, every four weeks, every five weeks, every six weeks, every seven weeks, every eight weeks or longer. In specific examples, the antibody is given to the subject at about 300 mg every two weeks. In other specific examples, the antibody is given to the subject at about 300 mg every four weeks.

In some embodiments, the therapeutically or prophylactically effective amount of the antibody (e.g., DX-2930) can be about 150 mg or 300 mg and is administered to a subject that is less than 18 years old every week, every two weeks, every three weeks, every four weeks, every five weeks, every six weeks, every seven weeks, every eight weeks or longer. In specific examples, the antibody is given to the subject at about 300 mg every two weeks. In other specific examples, the antibody is given to the subject at about 300 mg every four weeks.

In some embodiments, the therapeutically or prophylactically effective amount of the antibody (e.g., DX-2930) can be about 150 mg or 300 mg and is administered every two weeks or every four weeks. In some embodiments, the therapeutically or prophylactically effective amount of the antibody (e.g., DX-2930) can be 300 mg and is administered to a subject every two weeks. In some embodiments, the therapeutically or prophylactically effective amount of the antibody (e.g., DX-2930) can be 300 mg and is administered to the subject every four weeks. In some embodiments, the therapeutically or prophylactically effective amount of the antibody (e.g., DX-2930) can be 150 mg and is administered to the subject every four weeks. In some embodiments, the therapeutically or prophylactically effective amount is administered at least two times, at least three times, at least four times, at least five times, at least six times, at least seven times, at least eight times, at least nine times, at least ten times, at least eleven times, at least twelve time, at least thirteen times, or more. In some embodiments, the first treatment period is 26 weeks. In some embodiments, the therapeutically or prophylactically effective amount is 150 mg and is administered to the subject every four weeks (e.g., every four weeks for 26 weeks, resulting in delivery of 7 doses total). In some embodiments, the therapeutically or prophylactically effective amount is 300 mg and is administered to the subject every two weeks (e.g., every two weeks for 26 weeks, resulting in delivery of 13 doses total). In some embodiments, the therapeutically or prophylactically effective amount is 300 mg and is administered to the subject every four weeks (e.g., every four weeks for 26 weeks, resulting in delivery of 7 doses total).

In one example, the first treatment period is 26 weeks and the antibody is administered on day 0, day 28, day 56, day 84, day 112, day 140, and day 168. In another example, the first treatment period is 26 weeks and the antibody is administered on day 0, day 14, day 28, day 42, day 56, day 70, day 84, day 98, day 112, day 126, day 140, day 154, and day 168. It would have been understood by those skilled in the art that the listed treatment schedule allows for a ±4 day (e.g., ±3 days, ±2 days, or ±1 day) window. For example, a dose given at day 10-18 would be encompassed by the dose of day 14 noted above.

In some embodiments, a therapeutically or prophylactically effective amount is administered in a dosage regimen during a second treatment period following the first treatment period. In some embodiments, the therapeutically or prophylactically effective amount is different in the first treatment period and the second treatment period. In some embodiments, the therapeutically or prophylactically effective amount for the second treatment period is about 300 mg. During this period, the antibody may be administered in multiple doses of about 300 mg, such as 300 mg administered every two weeks. In some embodiments, in the second treatment period, the multiple doses of the antibody are administered at least two times, at least three times, at least four times, at least five times, at least six times, at least seven times, at least eight times, at least nine times, at least ten times, at least eleven times, at least twelve time, at least thirteen times. In some embodiments, the second treatment period is 26 weeks. In some embodiments, the antibody is administered at a dose of about 300 mg every two weeks for 26 weeks (e.g. resulting in delivery of 13 doses). In some embodiments, the single first dose of the second treatment period is administered about two weeks after the last dose of the first treatment period.

In any of the embodiments described herein, the timing of the administration of the antibody is approximate and may include the three days prior to and three days following the indicated day (e.g., administration every two weeks encompasses administration on day 11, day 12, day 13, day 14, day 15, day 16, or day 17).

In some embodiments, an antibody as described herein is administered in a single dose of about 300 mg to a subject who has undergone a prior HAE treatment (a first treatment), such as a multi-dose treatment with the same anti-pKal antibody as described herein (e.g., DX-2930). If the subject experiences a HAE attack after the single dose, the subject can be treated by the antibody for multiple doses at about 300 mg every two weeks for a suitable period, for example, 26 weeks. In some embodiments, the first of the multiple doses is administered within one week of the HAE attack (e.g., within 1 day, 2, days, 3 days, 4 days, 5 days, 6 days, or 7 days of the HAE attack). In some embodiments, the antibody is administered at least two times, at least three times, at least four times, at least five times, at least six times, at least seven times, at least eight times, at least nine times, at least ten times, at least eleven times, at least twelve time, at least thirteen times, or more.

The prior HAE treatment can involve the same antibody as described herein (e.g., DX-2930). In some embodiments, the prior HAE treatment may involve multiple doses of DX-2930 every two weeks or every four weeks. In some embodiments, DX-2930 is given to the subject (e.g., subcutaneously) at 150 mg every four weeks, at 300 mg every two weeks, or at 300 mg every four weeks. In one example, the subject was previously administered the antibody every two weeks or four weeks for 26 weeks prior to administration of the single dose of the antibody. In some embodiments, the multiple doses of the antibody of the prior treatment are administered at least two times, at least three times, at least four times, at least five times, at least six times, at least seven times, at least eight times, at least nine times, at least ten times, at least eleven times, at least twelve time, at least thirteen times. In some embodiments, the antibody was previously administered to the day 0, day 28, day 56, day 84, day 112, day 140, and day 168. In some embodiments, the single dose of about 300 mg of the antibody is administered about two weeks after the last dose of the previous treatment. In one example, the single dose of the second treatment period is administered on day 182 of the first treatment period.

In any of the embodiments described herein, the timing of the administration of the antibody is approximate and includes the three days prior to and three days following the indicated day (e.g., administration every two weeks encompasses administration on day 11, day 12, day 13, day 14, day 15, day 16, or day 17).

In some embodiments, prior to administering an antibody according to any of the methods described herein, the subject may be evaluated to establish a baseline rate of HAE attacks. Such an evaluation period may be referred to as a “run-in period.” In some embodiments, the baseline rate of HAE attacks must meet or exceed a minimum number of HAE attacks in a given time period. In one example, the subject experiences at least one HAE attack in a four week run-in period prior to the first administration of the antibody. In another example, the subject experiences between 1 and less than 2 attacks per month in a four week run-in period prior to the first administration of the antibody. In another example, the subject experiences between 2 and less than 3 attacks per month in a four week run-in period prior to the first administration of the antibody. In another example, the subject experiences 3 or more attacks per month in a four week run-in period prior to the first administration of the antibody. In another example, the subject experiences at least two HAE attacks in an eight week run-in period prior to the first administration of the antibody. In yet another example, the subject experiences an average of at least one HAE attack per month.

In some embodiments, the therapeutically or prophylactically effective amount of the antibody (e.g., DX-2930) can be about 150 mg or 300 mg and is administered to a subject that has experienced between 1 and less than 2 HAE attacks per month in a run-in period prior to the first administration of the antibody, every two weeks, every three weeks, every four weeks, every five weeks, every six weeks, every seven weeks, every eight weeks or longer. In some embodiments, the therapeutically or prophylactically effective amount of the antibody (e.g., DX-2930) can be about 150 mg or 300 mg and is administered to a subject that has experienced between 2 and less than 3 HAE attacks per month in a run-in period prior to the first administration of the antibody, every two weeks, every three weeks, every four weeks, every five weeks, every six weeks, every seven weeks, every eight weeks or longer. In some embodiments, the therapeutically or prophylactically effective amount of the antibody (e.g., DX-2930) can be about 150 mg or 300 mg and is administered to a subject that has experienced more than 3 HAE attacks per month in a run-in period prior to the first administration of the antibody, every two weeks, every three weeks, every four weeks, every five weeks, every six weeks, every seven weeks, every eight weeks or longer.

In some embodiments, administering an antibody according to any of the methods described herein results in a reduction of the average rate of HAE attacks in a subject. In some embodiments, a percent reduction of the average rate of HAE attacks after administering an antibody according to any of the methods described herein may be determined relative to a rate of HAE attacks in subjects who did not receive the antibody (e.g., subjects that were administered a placebo). In some embodiments, the percent reduction of the average rate of HAE attacks may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to a rate of HAE attacks in subjects who did not receive the antibody (e.g., subjects that were administered a placebo).

Any of the subjects described herein may have undergone prior treatment of HAE, such as a prophylactic or therapeutic treatment of HAE. Aspects of the present disclosure also provide methods of administering an antibody as described herein (e.g., DX-2930) to a subject that has received one or more prior treatment for HAE. In some embodiments, the prior treatment of HAE is a treatment that involves an antibody described herein (e.g., DX-2930). In some embodiments, the subject was previously administered multiple doses of DX-2930 every two weeks or every four weeks. In some embodiments, the subject was previously administered DX-2930 at 150 mg every two weeks. In some embodiments, the subject was previously administered DX-2930 at 300 mg every two weeks. In some embodiments, the subject was previously administered DX-2930 at 300 mg every four weeks. In some embodiments, the multiple doses of the antibody of the prior treatment are administered at least two times, at least three times, at least four times, at least five times, at least six times, at least seven times, at least eight times, at least nine times, at least ten times, at least eleven times, at least twelve time, at least thirteen times.

In some embodiments, the subject has received one or more prior treatment for HAE, such as a long term prophylactic treatment, which may involve any of the therapeutic agent for HAE known in the art. Exemplary anti-HAE agents include, but are not limited to, C1-inhibitors (e.g., Cinryze®, Berinert®, or Ruconest®), plasma kallikrein inhibitors (e.g., Kalbitor®), bradykinin receptor inhibitors (e.g., Firazyr®), attenuated androgens (e.g., danazol), and anti-fibrinolytics (e.g., traexamic acid). In some embodiments, the subject has received treatment with a C1-inhibitor prior to the first treatment period. In some examples, a subject may undergo a tapering period before receiving the anti-pKal antibody treatment as described herein. A tapering period refers to a period, prior to the anti-pKal antibody treatment, during which a subject who is on an anti-HAE treatment (e.g., C1-INH, oral androgen, and/or oral anti-fibrinolytics) gradually reduces the dosage, frequency, or both of the anti-HAE agent such that the subject can gradually transit from the prior HAE treatment to the anti-pKal antibody treatment as described herein. In some embodiments, the tapering involving a gradual or step-wise method of reducing the dosage of the prior treatment and/or the frequency with which the prior treatment is administered. The tapering period may last 2-4 weeks and can vary based on factors of an individual patent. In some examples, the prior treatment terminates before the anti-pKal antibody treatment starts. In other examples, the prior treatment may terminate within a suitable timeframe (e.g., 2 weeks, 3 weeks, or 4 weeks) after the subject is given his or her first dose of the anti-pKal antibody.

Alternatively, a subject who is on a prior HAE treatment may be transitioned to the anti-pKal antibody treatment as described herein directly without the tapering period.

In some embodiments, the therapeutically or prophylactically effective amount of the antibody (e.g., DX-2930) can be about 150 mg or 300 mg and is administered to a subject that has received one or more prior treatments for HAE, every two weeks, every three weeks, every four weeks, every five weeks, every six weeks, every seven weeks, every eight weeks or longer.

In other embodiments, the subject is free of any prior treatment of HAE before the first treatment, first treatment period, and/or the follow-on single and multiple dose treatments as described herein (the second treatment period). In some embodiments, the subject is free of any treatment other than with the antibodies described herein during the first treatment period and/or during the second treatment period. In some embodiments, the subject is free of any prior treatment of HAE for at least two weeks (e.g., at least two, three, four, five weeks or more) before the first treatment or first treatment period, during the first treatment or first treatment period, and/or during the second treatment period. In some embodiments, the subject is free of long-term prophylaxis for HAE (e.g., C1 inhibitor, attenuated androgens, anti-fibrinolytics) for at least the two weeks prior to the first treatment or first treatment period, during the first treatment period, and/or during the second treatment period. In some embodiments, the subject is free of an HAE treatment involving an angiotensin-converting enzyme (ACE) inhibitor for at least the four weeks prior to the first treatment or first treatment period, during the first treatment period, and/or during the second treatment period. In some embodiments, the subject is free of an estrogen-containing medication for at least the four weeks prior to the first treatment or first treatment period, during the first treatment period, and/or during the second treatment period. In some embodiments, the subject is free of androgens (e.g. stanozolol, danazol, oxandrolone, methyltestosterone, testosterone) for at least the two weeks prior to the first treatment or first treatment period, during the first treatment period and/or during the second treatment period.

Any of the methods described herein may further comprise monitoring the patient for side effects (e.g., elevation of creatine phosphatase levels) and/or inhibition levels of pKal by the antibody (e.g., serum or plasma concentration of the antibody or the pKal activity level) before and after the treatment or during the course of treatment. If one or more adverse effect is observed, the dose of the antibody might be reduced or the treatment might be terminated. If the inhibition level is below a minimum therapeutic level, further doses of the antibody might be administered to the patient. Patients may also be evaluated for the generation of antibody against the administered antibody; activity of C1-inhibitor, C4, and/or C1q; quality of life; incidence of any HAE attacks, health-related quality of life, anxiety and/or depression (e.g., Hospital Anxiety and Depression Scale (HADS)), work productivity (e.g., Work Productivity and Activity Impairment Questionnaire (WPAI)), preference of the subcutaneous administration of the antibody (e.g., D-2930) relative to other injectibles, quality of life (e.g., angioedema-quality of life (AE-QOL), EuroQoL Group 5-dimension report).

In some embodiments, the plasma or serum concentration of the antibody (e.g., DX-2930) may be measured during the course of the treatment (e.g., after the initial dosage) for assessing the efficacy of the treatment. If the plasma or serum concentration of the antibody is lower than about 80 nM, a follow-up dosage may be needed, which may be the same or higher than the initial dosage. The plasma or serum concentration of the antibody may be measured by determining the protein level of the antibody in a plasma or serum sample obtained from the subject, e.g., by an immune assay or MS assay. The plasma or serum concentration of the antibody may also be measured by determining the inhibitory level of pKal in a plasma or serum sample obtained from a subject treated with the antibody. Such assays may include the synthetic substrate assay or the Western blot assay for measuring cleaved kininogen as described herein.

Alternatively or in addition, the plasma or serum level of creatine kinase and/or one or more coagulation parameters (e.g., activated partial thromboplastin time (aPTT), prothrombin time (PT), bleeding events) can be monitored during the course of the treatment. If the plasma or serum level of creatine kinase is found to elevate during the treatment, the dosage of the antibody may be reduced or the treatment may be terminated. Similarly, if one or more coagulation parameters are found to be significantly affected during the treatment, the dosage of the antibody may be modified or the treatment may be terminated.

In some embodiments, an optimal dosage (e.g., optimal prophylactic dosage or optimal therapeutic dosage) of the antibody (e.g., DX-2930) may be determined as follows. The antibody is given to a subject in need of the treatment at an initial dose. The plasma concentration of the antibody in the subject is measured. If the plasma concentration is lower than 80 nM, the dose of the antibody is increased in a subsequent administration. A dosage of the antibody that maintains the antibody plasma concentration above about 80 nM can be chosen as the optimal dosage for the subject. The creatine phosphokinase level of the subject can be monitored during the course of treatment and the optimal dosage for that subject can be further adjusted based on the creatine phosphokinase level, e.g., the dosage of the antibody might be reduced is elevation of creatine phosphokinase is observed during treatment.

(iii) Combination Therapies

An antibody as described herein (e.g., DX-2930) can be administered in combination with one or more of the other therapies for treating a disease or condition associated with plasma kallikrein activity, e.g., a disease or condition described herein. For example, an antibody as described herein (e.g., DX-2930) can be used therapeutically or prophylactically (e.g., before, during, or after the course of treatment) with another anti-plasma kallikrein Fab or IgG (e.g., another Fab or IgG described herein), another plasma kallikrein inhibitor, a peptide inhibitor, small molecule inhibitor, or surgery. Examples of plasma kallikrein inhibitors that can be used in combination therapy with a plasma kallikrein binding antibodies described herein include plasma kallikrein inhibitors described in, e.g., WO 95/21601 or WO 2003/103475.

One or more plasma kallikrein inhibitors can be used in combination with an antibody as described herein (e.g., DX-2930). For example, the combination can result in a lower dose of the inhibitor being needed, such that side effects are reduced.

An antibody as described herein (e.g., DX-2930) can be administered in combination with one or more current therapies for treating HAE. For example, DX-2930 antibody can be co-used with a second anti-HAE therapeutic agent such as ecallantide, a C1 esterase inhibitor (e.g., CINRYZE™), aprotinin (TRASYLOL®), and/or a bradykinin B2 receptor inhibitor (e.g., icatibant (FIRAZYR®)).

The term “combination” refers to the use of the two or more agents or therapies to treat the same patient, wherein the use or action of the agents or therapies overlaps in time. The agents or therapies can be administered at the same time (e.g., as a single formulation that is administered to a patient or as two separate formulations administered concurrently) or sequentially in any order. Sequential administrations are administrations that are given at different times. The time between administration of the one agent and another agent can be minutes, hours, days, or weeks. The use of a plasma kallikrein binding antibody described herein can also be used to reduce the dosage of another therapy, e.g., to reduce the side effects associated with another agent that is being administered. Accordingly, a combination can include administering a second agent at a dosage at least 10, 20, 30, or 50% lower than would be used in the absence of the plasma kallikrein binding antibody. In some embodiments, a subject can be given a C1-inhibitor as a loading IV dose or SC dose simultaneously with the first dose of an anti-pKal antibody (e.g., DX-2930) as described herein. The subject can then continue with the anti-pKal antibody treatment (without further doses of the C1-inhibitor).

A combination therapy can include administering an agent that reduces the side effects of other therapies. The agent can be an agent that reduces the side effects of a plasma kallikrein associated disease treatment.

(iv) Assays for Assessing a Treatment Regimen

Also within the scope of the present disclosure are assay methods for assessing efficacy of any of the treatment methods described herein. In some embodiments, the plasma or serum concentration of one or more biomarkers (e.g., 2-chain HMWK) associated with HAE may be may be measured prior to and/or during the course of the treatment (e.g., after the initial dosage) for assessing the efficacy of the treatment. In some embodiments, the plasma or serum concentration (level) of one or more biomarkers associated with HAE obtained at a time point after administration of a dosage is compared to the concentration of the biomarker in a sample obtained at an earlier time point after administration of a dosage or prior to administration of the initial dosage. In some embodiments, the biomarker is 2-HMWK.

The level of the biomarker may be measured by detecting the biomarker in a plasma or serum sample obtained from the subject, e.g., by an immunoassay, such as Western blot assay or ELISA, using an antibody that specifically detects the biomarker. In some embodiments, the level of 2-HWMK in a plasma or serum sample obtained from the subject is assessed by an immunoassay. Antibodies for use in immunoassays for the detection of 2-HWMK are known in the art and selection of such an antibody for use in the methods described herein will be evident to one of ordinary skill in the art.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Efficacy and Safety of DX-2930 Treatment In Human Patient Subpopulations

Lanadelumab is a sterile, preservative-free solution for injection, pH 6.0. The active ingredient, antibody DX-2930, is formulated using the following compendial components: 30 mM sodium phosphate dibasic dihydrate, 19.6 mM citric acid monohydrate, 50 mM L-histidine, 90 mM sodium chloride, 0.01% Polysorbate 80. Each vial contains a nominal concentration of 150 mg DX-2930 active ingredient in 1 mL solution. The test product is administered by subcutaneous (SC) injection into the upper arm in a blinded manner.

Placebo consists of the inactive formulation of the test product: 30 mM sodium phosphate dibasic dihydrate, 19.6 mM citric acid monohydrate, 50 mM L-histidine, 90 mM sodium chloride, pH 6.0 with 0.01% Polysorbate 80. Placebo doses were administered to subjects randomized to the placebo treatment arm and in between doses of DX-2930 for subjects randomized to the 300 mg or 150 mg DX-2930 every 4 weeks treatment arms.

Patients≥12 years old with HAE type I/II and ≥1 attack/month at baseline were randomized 2:2:2:3 to lanadelumab 150 mg every 4 weeks (q4 wks), 300 mg q4 wks, 300 mg q2 wks, or placebo. Exploratory analyses were planned for subgroups with adequate numbers of patients for Poisson regression.

The following primary and secondary efficacy endpoints were evaluated from Day 14 through Day 182. The primary endpoint of the study was the number of HAE attacks and average rate of HAE attacks. Secondary endpoints included, in rank order:

1. Number of HAE attacks requiring acute treatment

2. Number of moderate to severe HAE attacks

### Exploratory Efficacy Endpoints

1. Time to first attack after day 14, i.e., duration that a subject was attack-free after day 14 until their first attack.

2. Number per week of high-morbidity HAE attacks; a high-morbidity HAE attack is defined as any attack that has at least one of the following characteristics: severe, results in hospitalization (except hospitalization for observation<24 hours), hemodynamically significant (systolic blood pressure<90, requires IV hydration, or associated with syncope or near-syncope) or laryngeal.

### Clinical Laboratory Tests

Patients involved in the clinical study were subjected to laboratory testing including general safety parameters (hematology, coagulation, urinalysis, and serum chemistry), serology, pregnancy tests, C1-INH functional assay, C4 assay, C1q assay, PK samples, plasma anti-drug antibody testing, and PD samples. All laboratory tests is performed using established and validated methods.

### Results

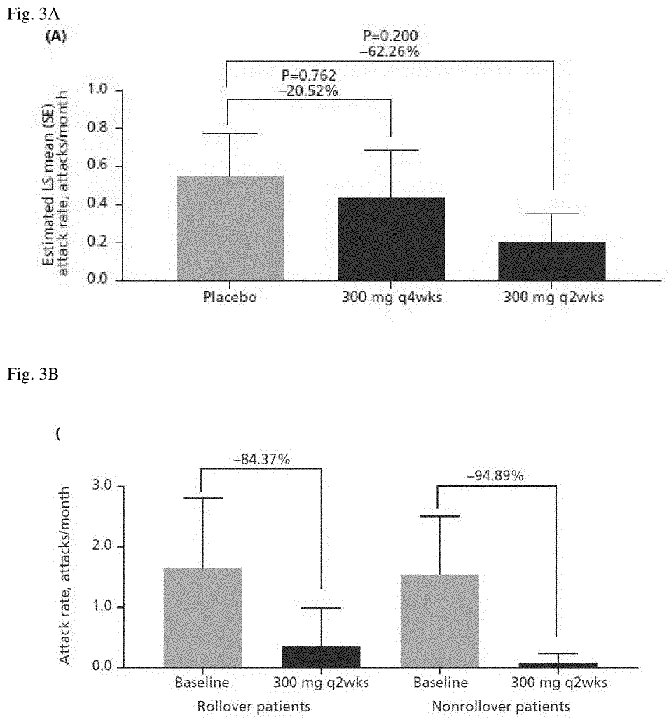

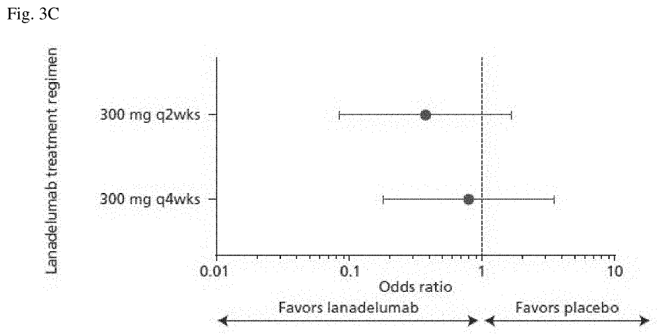

Overall, 125 patients were treated with lanadelumab (n=84) or placebo (n=41). The average rate of HAE attacks was determined for all patients and accordingly, for all patient subgroups. The average number of HAE attacks was used to determine percent reductions in the average rate of HAE attacks for patients who were administered DX-2930 relative to patients who received the placebo. HAE attack rates were consistently reduced with DX-2930 relative to placebo across all patients and patient subgroups. However, as shown in Table 2, greater percent reductions relative to placebo treatment, i.e., more therapeutically efficacious reductions, were observed for several patient subgroups when administered DX-2930 at 300 mg every two weeks relative to DX-2930 at 300 mg every four weeks (or DX-2930 at 150 mg every four weeks). Specifically, patients aged <18 years old who received DX-2930 at 300 mg every 4 weeks had a 20.5% reduction in HAE attack rate relative to placebo; patients aged <18 years old who received DX-2930 at 300 mg every 2 weeks had a further reduction in rate of about 42 percentage points (62.3%) (FIG. 3A). Patients aged 40-<65 years old who received DX-2930 at 300 mg every 4 weeks had a 71.5% reduction in HAE attack rate relative to placebo; patients aged 40-<65 years old who received DX-2930 at 300 mg every 2 weeks had a further reduction in rate of about 18 percentage points lower (89.8%). Female patients who received DX-2930 at 300 mg every 4 weeks had a 69.6% reduction in HAE attack rate relative to placebo; female patients who received DX-2930 at 300 mg every 2 weeks had a further reduction of about 16 percentage points (85.8%). Patients with a history of prior laryngeal attacks who received DX-2930 at 300 mg every 4 weeks had a 64.2% reduction in HAE attack rate relative to placebo; patients with a history of prior laryngeal attacks who received DX-2930 at 300 mg every 2 weeks had a further reduction of about 21 percentage points lower (85.7%).

TABLE 2
Percent Reductions in Patient Subgroups Relative to Placebo Treatment

| | DX-2930 at | DX-2930 at |
|---|---|---|
| | 300 mg every 4 | 300 mg every 2 |
| | weeks | weeks |
| Age < 18 years | 20.5% | 62.3% |
| Age 18-<40 years | 80.3% | 84.5% |
| Age 40-<65 years | 71.5% | 89.8% |
| Male | 82.4% | 90.3% |
| Female | 69.6% | 85.8% |
| Weight 50-<75 kg | 78.4% | 93.1% |
| Weight 75-<100 kg | 74.0% | 84.0% |
| Weight > 100 kg | 61.3% | 82.7% |
| HAE Type I | 73.4% | 87.8% |
| HAE Type II | 60.1% | 69.8% |
| Prior laryngeal attacks | 64.2% | 85.7% |
| No prior laryngeal attacks | 85.8% | 88.0% |

While all patients with HAE type I/II treated with lanadelumab 300 mg q2 wks or q4 wks all experienced clinically meaningful and persistent reductions in HAE attack rate compared with placebo, patient of certain subpopulations, e.g., female, patients who are <18 years old or between 40-65, and those who had at least one prior laryngeal attack, showed better treatment efficacy at 300 mg every two weeks.

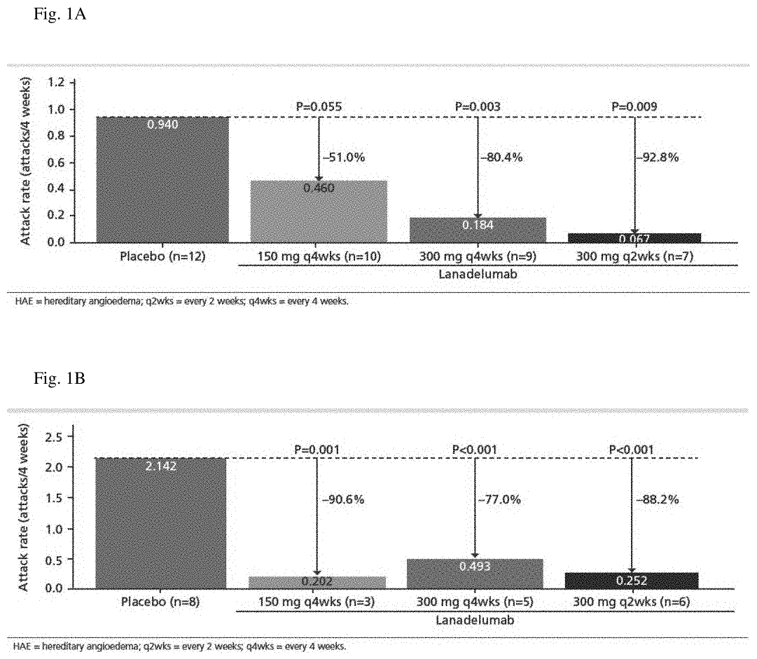

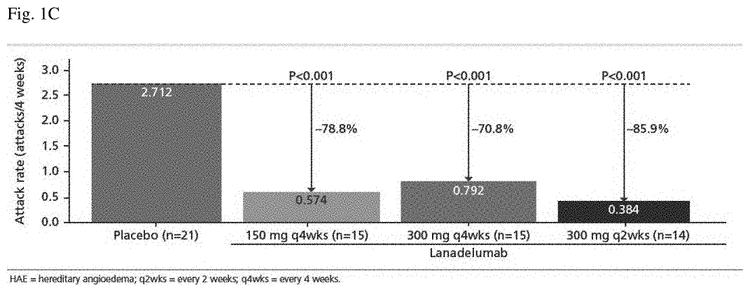

Patients were also stratified based on the HAE attacks during the run-in period to evaluate the efficacy of each of the lanadelumab treatment regimen in these patient subgroups. As shown in Tables 3-5 and FIGS. 1A-1C, each of the lanadelumab treatment regimens resulted in a significant reduction in HAE attack rate as compared to placebo across all subgroups.

TABLE 3
Patients with 1 to <2 attacks per month at run-in (n = 38)

| | | Lanadelumab | | |
|---|---|---|---|---|
| | | 150 mg | 300 mg | 300 mg |
| Characteristic | Placebo | q4wks | q4wks | q2wks |
| Run-in period HAE attack | | | | |
| rate | | | | |
| n | 12 | 10 | 9 | 7 |
| Mean (SD) | 1.22 (0.37) | 1.33 (0.45) | 1.30 (0.47) | 1.12 (0.36) |
| Median (min, max) | 1.0 (1.0, 1.9) | 1.0 (1.0, 1.9) | 1.0 (1.0, 1.9) | 1.0 (1.0, 1.9) |
| Treatment period HAE | | | | |
| attack rate | | | | |
| n | 12 | 10 | 9 | 7 |
| Mean (SD) | 0.94 (0.49) | 0.47 (0.69) | 0.19 (0.21) | 0.07 (0.08) |
| Median (min, max) | 1.02 (0.0, 1.7) | 0.15 (0.0, 1.8) | 0.15 (0.0, 0.5) | 0.0 (0.0, 0.2) |

TABLE 4
Patients with 2 to <3 attacks per month at run-in (n = 22)

| | | Lanadelumab | | |
|---|---|---|---|---|
| | | 150 mg | 300 mg | |
| Characteristic | Placebo | q4wks | q4wks | 300 mg q2wks |
| Run-in period HAE attack | | | | |
| rate | | | | |
| n | 8 | 3 | 5 | 6 |
| Mean (SD) | 2.31 (0.38) | 2.49 (0.43) | 2.27 (0.40) | 2.50 (0.41) |
| Median (min, max) | 2.14 (2.0, 2.9) | 2.67 (2.0, 2.8) | 2.0 (2.0, 2.9) | 2.59 (2.0, 2.9) |
| Treatment period HAE | | | | |
| attack rate | | | | |
| n | 8 | 3 | 5 | 6 |
| Mean (SD) | 2.12 (0.59) | 0.20 (0.35) | 0.49 (0.40) | 0.25 (0.29) |
| Median (min, max) | 2.12 (1.2, 2.9) | 0.0 (0.0, 0.6) | 0.62 (0.0, 0.9) | 0.15 (0.0, 0.6) |

TABLE 5
Patients with ≥3 attacks per month at run-in (n = 65)

| | | Lanadelumab | | |
|---|---|---|---|---|
| | | 150 mg | 300 mg | |
| Characteristic | Placebo | q4wks | q4wks | 300 mg q2wks |
| Run-in period HAE attack | | | | |
| rate | | | | |
| n | 21 | 15 | 15 | 14 |
| Mean (SD) | 6.27 (3.16) | 4.62 (1.25) | 5.63 (1.99) | 5.16 (2.06) |
| Median (min, max) | 5.0 (3.0, 14.7) | 4.0 (3.0, 6.7) | 5.33 (3.0, 10.5) | 4.08 (3.1, 9.0) |
| Treatment period HAE | | | | |
| attack rate | | | | |
| n | 21 | 15 | 15 | 14 |
| Mean (SD) | 3.45 (2.44) | 0.55 (9.64) | 0.89 (1.00) | 0.45 (0.65) |
| Median (min, max) | 2.30 (0.8, 8.3) | 0.30 (0.0, 2.0) | 0.46 (0.0, 2.9) | 0.15 (0.0, 1.8) |

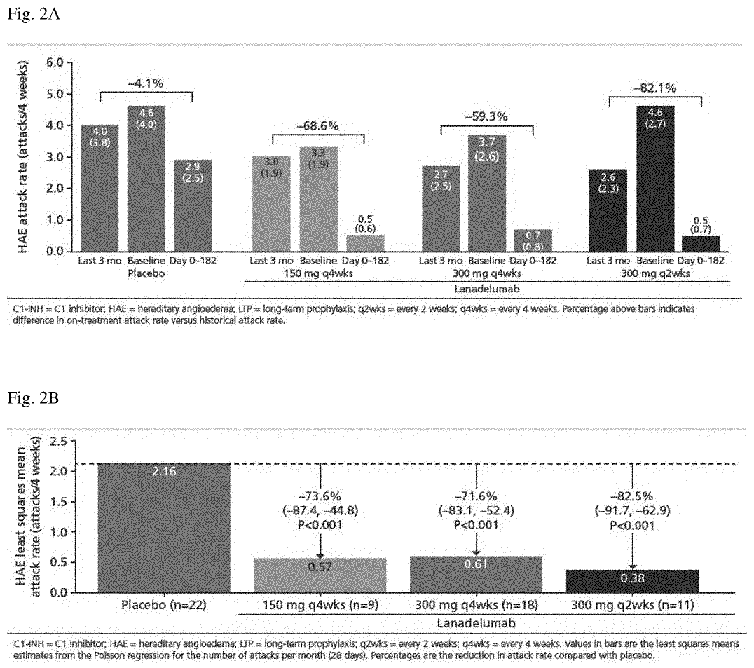

In patients who used only C1-inhibitor (C1-INH) as a long term prophylaxis, the attack rates at baseline increased relative to historical rates (during the last 3 months) during discontinuation of C1-INH per protocol (FIG. 2A). Attack rates during lanadelumab treatment were lower than historical attack rates. The attack rate decreased on average by 68.8%, 59.3%, and 82.1% during treatment with lanadelumab 150 mg q4 wks, 300 mg q4 wks, and 300 mg q2 wks, respectively, relative to historical attack rates while on long term prophylaxis.

There was a consistent treatment effect of lanadelumab in patients who used C1-INH only for prophylaxis and in patients who did not use long term prophylaxis when compared with placebo using the Poisson regression model (FIG. 2B). In patients who used C1-INH only for long term prophylaxis before administration of lanadelumab, the mean attack rate was significantly reduced by 73.6%, 71.6%, and 82.5% in the lanadelumab 150 mg q4 wks, 300 mg q4 wks, and 300 mg q2 wks regiments, respectively, versus placebo (P<0.001 for all comparisons).

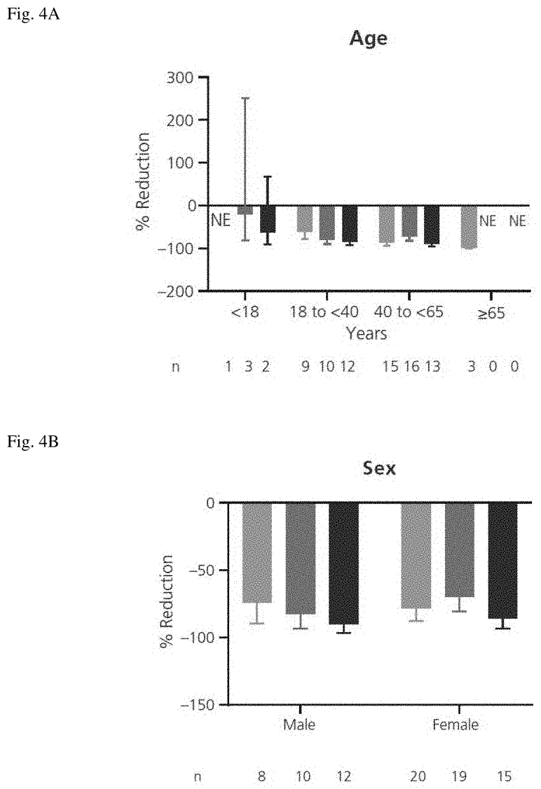

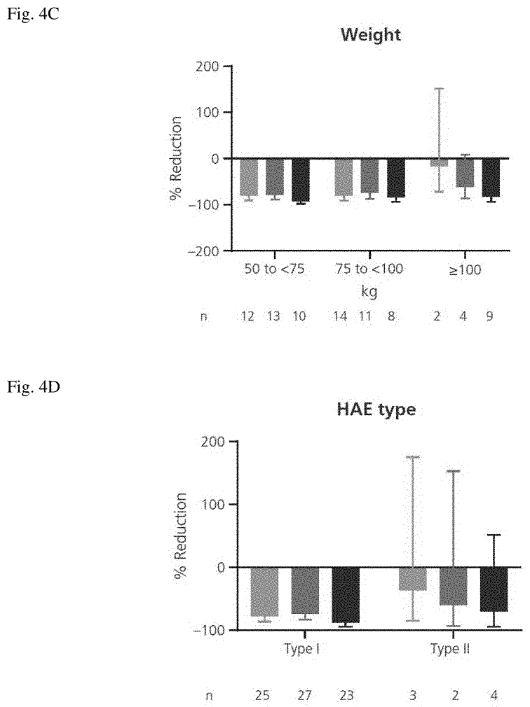

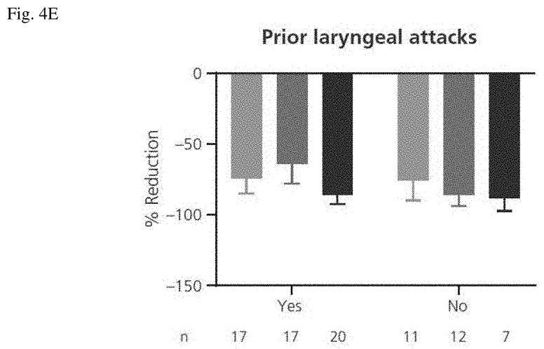

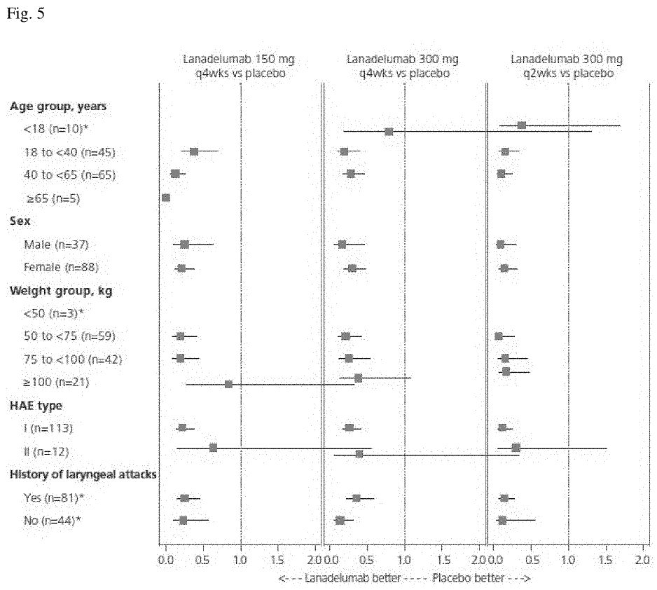

The percentage reduction in HAE attack rates in subjects that were administered lanadelumab at each of the dosing regimens compared to placebo was evaluated for subgroups of subjects, such as based on age, sex, weight, HAE type (e.g., Type I or Type II), and prior laryngeal attacks. FIGS. 4A-4E and 5.

Lanadelumab markedly suppressed pKal activation as shown by its effect on cHMWK levels. Optimal clinical responses with a fixed dose regimen of 300 mg every two weeks were observed in adolescents and adults across a large range of body weights.

### Example 2

### Efficacy and Safety of DX-2930 (Lanadelumab) Treatment in Human Adolescent Patients

The efficacy and safety of lanadelumab, a monoclonal antibody targeting plasma kallikrein, in adolescents with HAE with C1 inhibitor deficiency were investigated in this Phase 3 study and an open-label extension (OLE) study.

For the Phase 3 study, patients aged ≥12 years with ≥1 investigator-confirmed attack/4 weeks were randomized to placebo, or 150 mg every 4 weeks (150 mg q4 w), 300 mg q4 w, or 300 mg q2 w lanadelumab. In the Phase 3 study, 10 of 125 patients (8%) were adolescents (≥12 to <18 years of age). Before initiation of the Phase 3 study, 60.0% of patients received C1-INH only for long-term prophylaxis.

In general, rollover subjects in the open-label extension study were treated with lanadelumab following a treatment regimen of the Phase 3 trial (i.e., 150 mg every 4 weeks, 300 mg every 4 weeks, 300 mg every 2 weeks). In the open-label extension study, the subjects receive a single open-label dose of 300 mg lanadelumab administered subcutaneously on Day 0. The subject did not receive any additional lanadelumab doses until their first reported, and investigator-confirmed, HAE attack. Once a rollover subject reports his or her first HAE attack, the subject receives a second open-label dose of lanadelumab as soon as possible, with a minimum of 10 days between the first open-label dose and the second open-label dose. Following the second dose, rollover subjects continue to receive repeated subcutaneous administration of open-label 300 mg lanadelumab every 2 weeks for the remaining duration of the treatment period per the scheduled dosing. The treatment period lasts 350 days from the date of the first open-label dose.

Non-rollover subjects in the open-label extension study receive an open-label dose of 300 mg lanadelumab administered subcutaneously on Day 0 and continues to receive subcutaneous administrations of open-label 300 mg lanadelumab every 2 weeks throughout the duration of the treatment period per the scheduled dosing. A total of 26 doses are administered with the last dose administered at the Day 350 visit.

For rollover patients, 62.5% received C 1-INH only before initiation of the open-label extension study. For non-rollover adolescent patients, 61.6% received long-term prophylaxis therapy (C1-INH only or C1-INH and oral therapy) before initiation of the study (primarily C1-INH only; 46.2%). Monthly attack rate (MAR) and other treatment-emergent events (TEAEs) were recorded.

Three adolescent subjects had 13 non-serious treatment emergent adverse events (TEAEs). In the open label extension study, 21/212 patients (9.9%) were adolescents. Rollover patients (n=8) and non-rollover patients (n=13), respectively, had a mean (SD) monthly attack rate of 1.65 (1.158) and 1.54 (0.971) at baseline and 0.35 (0.635) and 0.07 (0.166) during the treatment period, i.e., a mean (SD) percent change of −84.371 (18.9415) and −94.893 (10.5230). Nine patients had 65 non-serious lanadelumab-related TEAEs.

The results from this study is provided in Table 6 below. Lanadelumab was found to be effective in reducing MAR and safe in adolescents with HAE.

TABLE 6
Percent Reductions in Adolescent Subjects between 12 years
and less than 18 years old.

| | | | Monthly attack rate |
|---|---|---|---|
| | | Monthly attack rate | during treatment |
| | Number of | during run-in | period (standard |
| | subjects | (standard deviation) | deviation) |
| Placebo | 4 | 1.825 (1.460) | 0.917 (0.992) |
| 150 mg every 4 | 1 | 1.000 | 0.000 |
| weeks | | | |
| 300 mg every 2 | 3 | 0.989 (0.020) | 0.304 (0.263) |
| weeks | | | |
| 300 mg every 2 | 2 | 1.948 (1.341) | 0.306 (0.433) |
| weeks | | | |

In the Phase 3 study, a lower least squares mean (SE) HAE attack rate was observed from day 0 to day 182 in patients treated with lanadelumab 300 mg q4 wks (n=3; 0.436 [0.253]) or lanadelumab 300 mg q2 wks (n=2; 0.207 [0.148]) compared with those who received placebo (n=4; 0.548 [0.224]). This was not estimated in the 150 mg q4 wks treatment arm as it included only 1 adolescent patient. The estimated least squares mean monthly attack rate ratio (versus placebo), with 95% CI, favored treatment with lanadelumab, particularly the 300 mg q2 wks dose regimen (FIG. 3C). In the open-label extension study, the mean (SD) percent change from baseline in mean monthly attack rate was 84.37 (18.94) for rollover patients (n=8; at the regular dosing stage) and −94.89 (10.52) for non-rollover patients (n=13; FIG. 3B).

In the Phase 3 study, 3 adolescent patients had 13 nonserious lanadelumab-related TEAEs (Table 7). The most common TEAEs that occurred in >1 patient during treatment with lanadelumab were injection site pain (3 patients) and rash (2 patients). In the open-label extension study, 9 patients had 65 nonserious lanadelumab-related TEAEs over a mean subject-time of 0.63 years. The most common TEAEs that occurred in >1 patient were injection site pain (9 patients), viral upper respiratory tract infection (3 patients), influenza (2 patients), pharyngitis streptococcal (2 patients), upper respiratory tract infection (2 patients), abdominal pain (2 patients), and headache (2 patients). Overall, the most common TEAE related to lanadelumab administration that was recorded in >1 patient was injection site pain (3 patients in the Phase 3 study and 8 patients in the OLE study; Table 7). These were similar to those identified in the overall population of the Phase 3 study.

In both the Phase 3 study and its OLE, there were no deaths or study discontinuations due to a TEAE.

TABLE 7
TEAEs (excluding HAE attacks) during the treatment period for adolescent patients
in the Phase 3 study and its OLE.

| | Phase 3 study | | | | | | | | | |
|---|---|---|---|---|---|---|---|---|---|---|
| | | | Lanadelumab | | | | | | | |
| | | | 150 mg | | 300 mg | | 300 mg | | | |
| | Placebo | | q4wks | | q4wks | | q2wks | | Total | |
| N | 4 | | 1 | | 3 | | 2 | | 6 | |
| | n (%) | m | n (%) | m | n (%) | m | n (%) | m | n (%) | m |
| Any TEAE | 2 (50.0) | 6 | 1 (100.0) | 3 | 2 (66.7) | 4 | 2 (100.0) | 23 | 5 (83.3) | 30 |
| Any related | 1 (25.0) | 1 | 1 (100.0) | 1 | 0 (0.0) | 0 | 2 (100.0) | 12 | 3 (50.0) | 13 |
| TEAE | | | | | | | | | | |
| Any serious | 0 (0.0) | 0 | 0 (0.0) | 0 | 0 (0.0) | 0 | 1 (50.0) | 1 | 1 (16.7) | 1 |
| TEAE* | | | | | | | | | | |
| Any severe | 0 (0.0) | 0 | 0 (0.0) | 0 | 0 (0.0) | 0 | 1 (50.0) | 1 | 1 (16.7) | 1 |
| TEAE* | | | | | | | | | | |
| Hospitalizations | 0 (0.0) | 0 | 0 (0.0) | 0 | 0 (0.0) | 0 | 1 (50.0) | 1 | 1 (16.7) | 1 |
| due to a TEAE | | | | | | | | | | |

| | Extension study | | |
|---|---|---|---|
| | Lanadelumab | | |
| | | Non- | |
| | Rollover | rollover | |
| | Patients | Patients | Total |
| N | 8 | 13 | 21 |

| | n (%) | m | n (%) | m | n (%) | m |
|---|---|---|---|---|---|---|
| Any TEAE | 7 (87.5) | 23 | 11 (84.6) | 68 | 18 (85.7) | 91 |
| Any related | 2 (25.0) | 15 | 7 (53.8) | 50 | 9 (42.9) | 65 |
| TEAE | | | | | | |
| Any serious | 0 (0.0) | 0 | 0 (0.0) | 0 | 0 (0.0) | 0 |
| TEAE* | | | | | | |
| Any severe | 0 (0.0) | 0 | 0 (0.0) | 0 | 0 (0.0) | 0 |
| TEAE* | | | | | | |
| Hospitalizations | 0 (0.0) | 0 | 0 (0.0) | 0 | 0 (0.0) | 0 |
| due to a TEAE | | | | | | |

HAE: hereditary angioedema;
m = number of events;
TEAE = treatment-emergent adverse event;
q2wks =every 2 weeks;
q4wks = every 4 weeks. TEAEs are shown during the treatment period (day 0 to day 182) for the phase 3 study.
*In the phase 3 study, no serious or severe TEAEs were related to lanadelumab. Serious TEAEs were defined as any TEAE that resulted in death, a life-threatening experience, non-pre-planned hospitalization, persistent/significant disability/incapacity, an important medical event, or an experience that was a congenital anomaly/birth defect. Severe TEAEs were TEAEs classified as severe (grade 3, led to marked limitation in activity with some assistance usually required, required medical intervention/therapy, and/or possible hospitalization) or life-threatening (grade 4, led to extreme limitation in activity with significant assistance required, significant medical intervention/therapy required and/or probable hospitalization/hospice care) by the investigator.

In conclusion, lanadelumab administration was well-tolerated and reduced the monthly attack rate adolescents subjects in the Phase 3 study and the open-label extension study.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for treating hereditary angioedema (HAE) attack or reducing the rate of HAE attack, the method comprising:
administering to a human subject in need thereof an antibody comprising heavy chain complementarity determining regions (CDRs) set forth as SEQ ID NOs: 5-7 and light chain CDRs set forth as SEQ ID NOs: 8-10 for a first treatment period;
wherein, in the first treatment period, the antibody is administered in multiple doses to the human subject at about 300 mg every two weeks; and
wherein the human subject has, is suspected of having, or is at risk for HAE and is:
(i) female;
(ii) less than 18 years old or between the ages of 40-65 years old;
(iii) has experienced at least one prior laryngeal HAE attack;
(iv) has had between 1 and 2, between 2 and 3, or more than 3 HAE attacks in the four weeks prior to the first dose of the first treatment period; and/or
(v) has received treatment with a C1-inhibitor prior to the first treatment period.

2. A method for treating hereditary angioedema (HAE) attack or reducing the rate of HAE attack, the method comprising administering to a human subject in need thereof an antibody comprising heavy chain CDRs set forth as SEQ ID NOs: 5-7 and light chain CDRs set forth as SEQ ID NOs: 8-10, wherein the human subject is
(i) an adolescent between the age of 12 and 18; and/or
(ii) has had between 2 and 3 or more than 3 HAE attacks in the four weeks prior to the first dose of the antibody, and
wherein the antibody is administered to the human subject at about 150 mg every four weeks, at about 300 mg every four weeks, or at about 300 mg every two weeks.

3. The method of claim 1, wherein the antibody is a full length antibody or an antigen-binding fragment thereof.

4. The method of claim 1, wherein the antibody comprises a heavy chain variable region set forth by SEQ ID NO: 3 and/or a light chain variable region set forth by SEQ ID NO: 4.

5. The method of claim 1, wherein the antibody comprises a heavy chain set forth by SEQ ID NO: 1 and a light chain set forth by SEQ ID NO: 2.

6. The method of claim 1, wherein the antibody is formulated in a pharmaceutical composition comprising a pharmaceutically acceptable carrier; optionally wherein the pharmaceutical composition comprises sodium phosphate, citric acid, histidine, sodium chloride, and polysorbate 80.

7. (canceled)

8. The method of claim 6, wherein the sodium phosphate is at a concentration of about 30 mM, the citric acid is at a concentration of about 19 mM, the histidine is at a concentration of about 50 mM, the sodium chloride is at a concentration of about 90 mM, and the polysorbate 80 is at about 0.01%.

9. The method of claim 1, wherein the antibody is administered subcutaneously.

10. The method of claim 1, wherein the human subject.

11. The method of claim 1, wherein the human subject has experienced at least two HAE attacks per year prior to the first treatment period; and/or the human subject has received one or more prior HAE treatments prior to the first treatment period.

12. (canceled)

13. The method of claim 11, wherein the prior HAE treatment comprises a C1-inhibitor, a plasma kallikrein inhibitor, a bradykinin receptor antagonist, an androgen, an anti-fibrinolytic agent, or a combination thereof; optionally wherein the prior HAE treatment comprises C1-INH, ecallantide, icatibant, danazol, tranexamic acid, or a combination thereof.

14. (canceled)

15. The method of claim 11, wherein the human subject has received one or more prior HAE treatments prior to the first treatment period and the method comprises a tapering period for the one or more prior HAE treatments; optionally wherein the tapering period is about 2-4 weeks.

16. (canceled)

17. The method of claim 13, wherein the human subject has received one or more prior HAE treatments prior to the first treatment period and the one or more prior HAE treatments terminate either before the first dose of the antibody or within three weeks after the first dose of the antibody.

18. The method of claim 1, wherein the human subject is free of prior HAE treatment; optionally wherein the human subject is free of prior HAE treatment at least two weeks before the first dose of the antibody.

19. (canceled)

20. The method of claim 1, wherein the human subject has had at least one HAE attack in the four weeks prior to the first dose of the first treatment period or at least two HAE attacks in the eight weeks prior to the first dose of the first treatment period.

21. The method of claim 1, wherein the method further comprises administering to the subject the antibody for a second treatment period after the first treatment period.

22. The method of claim 21, wherein the first dose of the second treatment period is about two weeks after the last dose of the first treatment period; and/or
wherein the second treatment period comprises one or more doses of the antibody at about 300 mg, optionally wherein the second treatment period comprises multiple doses of the antibody at about 300 mg every two weeks.

23. (canceled)

24. (canceled)

25. The method of claim 1, wherein the human subject is free of a long-term prophylaxis for HAE, or an HAE treatment involving an angiotensin-converting enzyme (ACE) inhibitor, an estrogen-containing medication, or an androgen prior to the first treatment period, during the first treatment period, and/or during the second treatment period.

26. The method of claim 1, further comprising:
(a) administering to the human subject in need thereof the antibody at a single dose of about 300 mg after the first treatment period; and
(b) further administering to the subject the antibody at one or more doses of about 300 mg, if the subject experiences an HAE attack after (a); optionally wherein the single dose of (a) and the first dose of (b) are at least 10 days apart.

27. The method of claim 26, wherein in step (b), the subject is administered the antibody for multiple doses at about 300 mg every two weeks; optionally wherein the first dose of step (b) is within one week after the HAE attack.

28.-29. (canceled)


SEQUENCE LISTING (10 sequences)

SEQ ID NO: 1 - protein, 469 aa, Artificial Sequence
  Synthetic polypeptide
MGWSCILFLVATATGAHSEVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 231 aa, Artificial Sequence
  Synthetic polypeptide
MGWSCILFLVATATGAHSDIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 122 aa, Artificial Sequence
  Synthetic polypeptide
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSS

SEQ ID NO: 4 - protein, 106 aa, Artificial Sequence
  Synthetic polypeptide
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIK

SEQ ID NO: 5 - protein, 5 aa, Artificial Sequence
  Synthetic polypeptide
HYIMM

SEQ ID NO: 6 - protein, 17 aa, Artificial Sequence
  Synthetic polypeptide
GIYSSGGITVYADSVKG

SEQ ID NO: 7 - protein, 13 aa, Artificial Sequence
  Synthetic polypeptide
RRIGVPRRDEFDI

SEQ ID NO: 8 - protein, 11 aa, Artificial Sequence
  Synthetic polypeptide
RASQSISSWLA

SEQ ID NO: 9 - protein, 7 aa, Artificial Sequence
  Synthetic polypeptide
KASTLES

SEQ ID NO: 10 - protein, 8 aa, Artificial Sequence
  Synthetic polypeptide
QQYNTYWT